(19) United States
(12) Patent Application Publication
(10) Pub. No.: US 2003/0087338 A1
(43) Pub. Date: May 8, 2003

(54) Adhesive DOPA-containing polymers and related methods of use

(76) Inventors: Phillip B. Messersmith, Clarendon Hills, IL (US); Kui Huang, Evanston, IL (US); Bruce P. Lee, Evanston, IL (US); Jeffrey Dalsin, Chicago, IL (US); Bi-Huang Hu, Chicago, IL (US); Jonathan Friedstat, Wilmette, IL (US)

Correspondence Address: REINHART BOERNER VAN DEUREN S.C., ATTN: LINDA GABRIEL, DOCKET COORDINATOR, 1000 NORTH WATER STREET, SUITE 2100, MILWAUKEE, WI, 53202, US

(21) Appl. No.: 10/199,960

(22) Filed: Jul. 19, 2002

Related U.S. Application Data
(60) Provisional application No. 60/373,919, filed on Apr. 19, 2002.
(60) Provisional application No. 60/306,750, filed on Jul. 20, 2001.

(51) Int. Cl. C12P 21/06; C08H 1/00

(52) U.S. Cl. 435/68.1; 527/200

(57) ABSTRACT

3,4-Dihydroxyphenyl-L-alanine (DOPA) is an unusual amino acid found in mussel adhesive proteins (MAPs) that form tenacious bonds to various substrates under water. DOPA is believed to be responsible for the adhesive characteristics of MAPs. This invention relates to a route for the conjugation of DOPA moieties to various polymeric systems, including but not limited to poly(ethylene glycol) or poly(alkylene oxide) systems such as poly(ethylene oxide)-poly(propylene oxide)-poly(ethylene oxide) (PEO-PPO-PEO) block copolymers.

The United States Government has certain rights to this invention pursuant to Grant No. DE13030 from the National Institutes of Health to Northwestern University.

This application claims priority benefit from United States provisional patent applications, serial numbers 60/306,750 and 60/373,919 filed, respectively, on Jul. 20, 2001 and Apr. 19, 2002, each of which is incorporated herein by reference in its entirety.

### BACKGROUND OF INVENTION.

Poly(ethylene oxide)-poly(propylene oxide)-poly(ethylene oxide) (PEO-PPO-PEO) triblock copolymers, known commercially under the Pluronic® trade name in the United States, are widely used in diverse industrial applications.¹ Recently, such poly(alkylene oxides)(PAOs) have attracted considerable interest in the biotechnological and pharmaceutical industry for their unique surfactant abilities, low toxicity, and minimal immune response.²⁻¹³ Aqueous solutions of Pluronic® PAO copolymers exhibit interesting temperature-induced aggregation phenomena as a result of the hydrophobic nature of the PPO block.^{14,15} At low temperature and concentration, these polymers exist in solution as dissolved monomers. Block copolymer micelles self-assemble or form under isothermal conditions when the copolymer concentration is increased, or at constant concentration when the temperature is increased. Concentrated solutions of certain PAOs, such as those available using materials manufactured by BASF under the trade name or designations F127 (PEO₁₀₀PPO₆₅PEO₁₀₀) and F68 (PEO₇₈PPO₃₀PO₇₈), exhibit sol-gel transitions when heated above ambient temperature, a property which is potentially useful for medical drug delivery applications.¹⁶⁻²⁰ For example, in-situ gelling materials are potentially useful as injectable carriers for drug delivery to mucosal surfaces,²⁰ i.e. the oral cavity and the respiratory, gastrointestinal, and reproductive tracts.

However, in such carrier/delivery use or other hydrogel applications, adhesion is a desirable characteristic. PAOs, like other polymeric systems, provide no particular benefit, in this regard. Typically, a separate component is mixed with such systems to provide the adhesive properties required. Accordingly, efforts have been ongoing to enhance the adhesive, especially bioadhesive, properties of such systems.

One strategy for enhancing the bioadhesive characteristics of polymers is to introduce biological moieties that are known to possess adhesive properties in nature. For example, mussel adhesive proteins (MAPs) are remarkable underwater adhesive materials which form tenacious bonds that anchor marine organisms to the substrates upon which they reside.^{21,22} The protein adhesives are secreted as fluids which undergo an in-situ crosslinking or hardening reaction leading to the formation of a solid adhesive plaque.²¹⁻²⁴ One of the unique structural features of MAPs is the presence of 3-(3,4-dihydroxyphenyl)-L-alanine (DOPA), an amino acid which is believed to be responsible for both adhesion and crosslinking characteristics of MAPs.²⁵-28 DOPA is not a genetically encoded amino acid; instead, DOPA residues are formed by post translational enzymatic modification of Tyr-containing proteins by tyrosinase enzyme. Further oxidation of DOPA to DOPA-quinone can lead to crosslinking of the protein, whereas the cathechol form of DOPA is believed to be responsible for adhesion to substrates.²⁷

Recently, DOPA-containing synthetic polypeptides have been chemically synthesized by copolymerization of N-carboxyanhydride monomers of lysine and DOPA.29 The water soluble polypeptides were found to form crosslinked gels in the presence of oxidizing agents, and adhesion to various substrates was observed. Experimental evidence to date suggests, however, that the oxidative crosslinking agents used result in reduced adhesive potential, such reduction attributable to the DOPA oxidation. In addition, such oxidizing agents are physiologically or biologically harmful and could not be used in medical/dental treatment or pharmaceutical formulations.

Accordingly, there is an ongoing need in the art relating to bioadhesive polymers, particularly bioadhesive polymers having the ability to form hydrogels in-situ. A concurrent need relates to the preparation and design of a polymeric structure whereby DOPA can be introduced without harmful biological/physiological effect or loss of adhesive properties.

Another concern in the art relates to the use of polymeric materials for surface modification. Such a concern can arise in the context of the performance of an implanted biomaterial, in particular the interface between such a material and the biological environment. In the medical arena, the physical or chemical immobilization of polymers on material surfaces has been widely employed as a strategy to limit adsorption of proteins and cells to such surfaces. Control of protein and cell adhesion is critical to the performance of biosensors, implantable medical devices, and in the rapidly emerging area of nanoparticle therapies and diagnostics.

Poly(ethylene glycol) (PEG) is one such polymer which can be physically or chemically immobilized on a material surface, as part of an anti-fouling strategy to limit protein adsorption and, in turn, control the behavior of cells at material/tissue interfaces. PEG is one of numerous polymers which has also been used for steric stabilization of other molecules or particles in solution. Many nanoparticles aggregate and precipitate out of solution, especially in biological fluids. A polymeric surface layer has been shown to stabilize the nanoparticles in solution, presumably in a manner similar to that responsible for the anti-fouling effects observed on macroscopically flat material surfaces.

Polymer modification of material surfaces, whether macroscopically flat or nanoparticulate, is currently tailored for each type of material, requiring a number of different chemical strategies. For example, noble metal surfaces are typically modified using thiol chemistry, whereas metal oxides are modified using silane coupling techniques. Other modification routes are hindered by reliance on expensive instrumentation, complex synthetic procedures, or both. No surface modification currently exists for wide range application to a variety of different material surfaces.

Accordingly, there is also an ongoing need in the art relating to polymeric surface modification, in particular a surface-bound polymeric material providing steric resistance to particulate aggregation and/or anti-fouling properties. A concurrent need relates to the preparation and design of a polymeric system adhesive to a wide variety of material and/or nanoparticulate surfaces.

As indicated by the foregoing and several subsequent notations, these and other aspects of the prior art can be found in the following:

(1) Alexandridis, P. Curr. Opin. Colloid Interface Sci. 1997, 2, 478.

(2) Nace, V. M. Nonionic Surfactants; Marcel-Dekker: New York, 1996.

(3) Tan, J. S.; Butterfiled, D. E.; Voycheck, C. L.; Caldwell, K. D.; Li, J. T. Biomaterials 1993, 14, 823.

(4) Deible, C. R.; Beckman, E. J.; Russell, A. J.; Wagner, W. R. J Biomed. Mater. Res. 1998,41,251.

(5) Muller, R. H.; Ruhl, D.; Schulze-Forster, K. Eur. J Phar. Sci. 1997, 5, 147.

(6) Desal, S. D.; Blanchard, J. J Phar. Sci. 1998, 87, 226.

(7) O'keefe, H. J.; Grines, L. C.; Dewood, A. M.; Schaer, L. G.; Browne, K.; Magorien, D. R.; Kalbfleish, M. J.; Fletcher, O. W.; MBateman, M. T.; Gibbons, J. R. Am. J Cardiol. 1996, 78, 747.

(8) Palmer, J. S.; Cromie, W. J.; Lee, R. C. J. Urol. 1998, 159, 2136.

(9) Green, R. J.; Davies, M. C.; Roberts, C. J.; Tendler, S. J. B. J. Biomed. Mater. Res. 1998, 42, 165.

(10) Davis, S. S.; Illum, L. Biomaterials 1988, 9, 111.

(11) Jewell, R. C.; Khor, S. P.; Kisor, D. F.; LaCroix, K. A. K. J. Phar. Sci. 1997, 86, 808.

(12) Hillery, A. M.; Florence, A. T. Int. J Pharm. 1996, 132, 123.

(13) Lemieux, P.; Vinogradov, S. V.; Gebhart, C. L.; Guerin, N.; Paradis, G.; Nguyen, H. K.; Ochietti, B.; Suzdaltseva, Y. G.; Bartakova, E. V.; Bronich, T. K.; ST-Pierre, Y.; Alakhov, V. Y.; Kabanov, A. V. J. of Drug Targeting 2000, 8, 91.

(14) Hvidt, S.; Jørgensen, E. B.; Brown, W.; Schillen, K. J. Phys. Chem. 1994, 98, 12320.

(15) Nyström, B.; Walderhaug, H. J. Phys. Chem. 1996, 100, 5433.

(16) Bromberg, L. E.; Ron, E. S. Adv. Drug Delivery Rev. 1998, 31, 197.

(17) Yang, L.; Alexandridis, P. Curr. Opin. Colloid Interface Sci. 2000, 5, 132.

(18) Barichello, J. M.; Morishita, M.; Takayama, K.; Nagai, T. Int. J of Pharm. 1999, 184, 189.

(19) Faulkner, D. M.; Sutton, S. T.; Hesford, J. D.; Faulkner, B. C.; Major, D. A.; Hellewell, T. B.; Laughon, M. M.; Rodeheaver, G. T.; Edlich, R. F. Am. J of Emergency Medicine 1996, 15, 20.

(20) Kellaway, I. W.; Warren, S. J. In Oral Mucosal Drug Delivery; Rathbone, M. J., Ed.; Marcel Dekker, Inc.: New York, 1996; Vol. 74, p 221.

(21) Waite, J. H. Chemtech 1987, 17, 692.

(22) Yamamoto, H. Biotech. Genet. Eng. Rev. 1996, 13, 133.

(23) Taylor, S. W.; Chase, D. B. Inorg. Chem. 1996, 35, 7572.

(24) Taylor, S. W.; Luther, G. W. I. Inorg. Chem. 1994, 35, 5819.

(25) Waite, J. H.; Tanzer, M. L. Biochem. & Biophy. Res. Comm. 1980, 96, 1554.

(26) Waite, J. H.; Tanzer, M. L. Science 1981, 212, 1038.

(27) Yu, M.; Hwang, J.; Deming, T. J. J. Am. Chem. Soc. 1999, 875, 301.

(28) Schnurrer, J.; Lehr, C. International J. of Pharm. 1996, 141, 251.

(29) Yu, M.; Deming, T. J. Macromolecules 1998, 31, 4739.

(30) Li, J.; Carlsson, J.; Lin, J.; Caldwell, K. D. Bioconj. Chem. 1996, 7, 592.

(31) Neff, J. A.; Caldwell, K. D.; Tresco, P. A. J. Biomed. Mater. Res. 1998, 40, 511.

(32) Ahmed, F.; Alexandridis, P.; Neelamegham Langmuir 2001, 17, 537.

(33) Chen, G.; Hoffman, A. S.; Kabra, B.; Rander, K. In Poly(ethylene glycol). chemistry and biological applications; Harris, J. M., Zalipsky, S., Eds.; American Chemical Society: Washington, D.C., 1997; Vol. 660, p 441.

(34) Patel, R. P.; Price, S. J. Org. Chem. 1965, 30, 3575.

(35) Arnow, L. E. J. Bio. Chem. 1937, 118, 531.

(36) Waite, J. H.; Benedict, C. V. Methods in Enzymology 1984, 107, 397.

(37) Bromberg, L. Langmuir 1998, 14, 5806.

(38) Miron, T.; Wilchek, M. Bioconj. Chem. 1993, 4, 468.

(39) Ghosh, A. K.; Duong, T. T.; Mckee, S. P.; Thompson, W. J. Tetra. Lett. 1992, 33, 2781.

(40) Baberjee, S. N.; Ressler, C. J. Org. Chem. 1976, 41, 3056.

(41) Yamamoto, Y.; Miyahera, Y. U.S. Pat. No. 5,098,999. 1992.

(42) Nakonieczna, L.; Przycgodzen, W.; Chimiak, A. Liebigs. Ann. Chem. 1994, 1055.

(43) Hu, B.; Messersmith, P. B. Tetra. Lett. 2000, 41, 5795.

(44) Cabana, A.; Aït-Kadi, A.; Juhász, J. J. Colloid and Interface Sci. 1997, 190, 307.

(45) Brown, W.; Schillen, K.; Almgren, M. J. Phys. Chem. 1991, 95, 1850.

(46) Yu, G.; Deng, Y.; Dalton, S.; Wang, Q.; Attwod, D.; Booth, C. J. Chem. Soc. Faraday Trans. 1992, 88, 2537.

(47) Alexandridis, P.; Nivaggioli, T.; Alan Hatton, T. Langmuir 1995, 11, 1468.

(48) Nyström, B.; Walderhaug, H.; Hansen, F. Faraday Discuss. 1995, 101, 335.

(49) Armstrong, J.; Chowdhry, B.; O'Brien, R.; Beeze, A.; Mitchell, J.; Leharne, S. J. Phys. Chem. 1995, 99, 4590.

(50) Schmolka, I. R. J. Biomed. Mater. Res. 1972, 6, 571.

(51) Bromberg, L. J. Phys. Chem. B 1998,102, 1956.

(52) Wanka, G.; Hoffman, H.; Ulbricht, W. Colloid & Polymer Science 1990, 268, 101.

### BRIEF DESCRIPTION OF THE DRAWINGS

FIG. 1 shows ¹H NMR spectra of Pluronic® F127, its carbonate intermediate (SC-PAO7) and DME-PAO7 in CDCl₃.

FIG. 2 provides differential scanning calorimetry thermograms of 30 wt % DME-PAO7, DOPA-PAO7, and unmodified Pluronic® F127 aqueous solutions. Arrows indicate the location of gelation endotherm.

FIG. 3 plots shear storage modulus, G′, of a 22 wt % DME-PAO7 aqueous solution as a function of temperature at 0.1 Hz and a strain of 0.45%. Shown in the inset is the rheological profile of a 22 wt % unmodified-Pluronic® F127 aqueous solution as a function of temperature.

FIG. 4 plots shear storage modulus, G′, of a 50 wt % DME-PAO8 aqueous solution as a function of temperature at 0.1 Hz and a strain of 0.45%. Shown in the inset is the rheological profile of a 50 wt % unmodified Pluronic® F68 aqueous solution as a function of temperature.

FIG. 5 plots storage moduli of DME-PAO8 aqueous solutions at 45 wt % and 50 wt %, respectively, as a function of temperature at 0.1 Hz and a strain of 0.45%.

FIGS. 6A and 6B show differential scanning calorimetry thermograms of (A) DOPA-PAO7 and (B) DME-PAO7 at different concentrations upon heating. Arrows indicate the location of gelation endotherm, observed only at higher polymer concentrations.

FIGS. 7A-C show high-resolution C(1s) XPS peaks for (A) un-modified Au, (B) m-PEG-OH, and (C) m-PEG-DOPA. A dramatic increase in the ether peak at 286.5 eV in (C) indicated the presence of PEG.

FIGS. 8A-C provide TOF-SIMS positive spectrum showing peaks representing catechol binding of gold. Spectra were normalized to Au peak (m/z˜197).

FIG. 9 provides TOF-SIMS spectra showing the positive secondary ion peak at mass m/z˜43 for unmodified Au substrate, Au exposed to mPEG-OH, mPEG-DOPA powder and Au exposed to mPEG-DOPA.

FIG. 10 shows TOF-SIMS spectra showing the positive secondary ion peaks for Au substrate chemisorbed with MPEG-DOPA. Catecholic binding of gold is observed at m/z˜225 (AuOC), 254 (AuOCCO), and 309. Less intense AuOₐC_{b} peaks are seen at m/z˜434, 450, 462, and 478. The periodic triplets seen in the m/z range 530-1150 correspond to Au bound to DOPA-(CH₂CH₂O)ₙ, where each subpeak is separated by 14 or 16 amu, representing CH₂, CH₂CH₂, and CH₂CH₂O in the PEG chain. This pattern was observed for n=1-15.

FIG. 11 shows SPR spectra of protein (0.1 mg/ml BSA) adsorption onto modified and unmodified gold surfaces. mPEG-DOPA and mPEG-MAPd modified surfaces exhibited reduced protein adsorption compared to bare gold and mPEG-OH modified surfaces.

FIG. 12 shows mPEG-DOPA concentration dependence of anti-fouling behavior. Gold surfaces were modified for 24 h at the MPEG-DOPA concentrations indicated, followed by analysis of the density and area of attached cells. (*=p<0.05, **=p<0.01, ***=p<0.001; black bars=total proj. area, gray bars=surface cell density)

FIG. 13 compares cell attachment and spreading on bare gold, mPEG-OH-treated gold, and gold modified with MPEG-DOPA 5K, mPEG-MAPd 2K, and mPEG-MAPd 5K under optimal conditions (50 mg/ml for 24h). (black bars=total proj. area, gray bars=surface cell density; ***=p<.001)

FIGS. 14 A-C are a series of SEM micrographs indicating the morphology of NIH 3T3 fibroblasts on (A)unmodified Au, (B)Au treated with mPEG-OH, and (C)mPEG-DOPA-modified Au. All treatments were at 50 mg/ml in DCM for 24 h.

FIG. 15 shows the UV/vis absorption spectrum of mPEG-DOPA stabilized magnetite nanoparticles suspended in several aqueous NaCl solutions at the concentrations as shown and plotted therein. Addition of NaCl did not induce nanoparticle precipitation.

FIG. 16 shows addition of salt to untreated Au nanoparticles induces aggregation. Shown are UV/vis scans of 10 nm untreated Au nanoparticles suspended in aqueous NaCl solutions (concentrations as shown and plotted therein). The attenuation and shift of the 520 nm absorption band with increasing NaCl concentration reflects aggregation of the nanoparticles.

FIG. 17 illustrates addition of salt to mPEG-DOPA stabilized Au nanoparticles does not induce aggregation. Shown are UV/vis scans of 10 nm mPEG-DOPA stabilized Au nanoparticles suspended in aqueous NaCl solutions (concentrations as shown and plotted therein). The lack of attenuation and shift of the 520 nm absorption band with increasing NaCl concentration reflects effective stabilization of the nanoparticles.

FIG. 18 plots the UV/vis absorption spectrum of mPEG-DOPA stabilized CdS nanoparticles suspended in aqueous NaCl solutions (concentrations as shown and plotted therein).

### SUMMARY OF THE INVENTION

In light of the foregoing, it is an object of the present invention to provide one or more catecholic and/or DOPA- or DOPA derivative-containing polymers and/or methods for their production, thereby overcoming various deficiencies and shortcomings of the prior art, including those outlined above. It will be understood by those skilled in the art that one or more aspects of this invention can meet certain objectives, while one or more other aspects can meet certain other objectives. Each objective may not apply equally, in all its respects, to every aspect of this invention. As such, the following objects can be viewed in the alternative with respect to any one aspect of this invention.

It is an object of the present invention to provide one or more polymers or co-polymers, preferably exhibiting various oxidation independent aggregation and/or gelation phenomena, having incorporated therein one or more DOPA or DOPA-derived residues or monomers.

It can also be an object of the present invention to provide one or more polymeric or co-polymeric materials, such as those described above, having incorporated therein one or more DOPA or DOPA-derived components with a structural composition maintaining the adhesive characteristics, of the corresponding DOPA residue. Accordingly, it can also be an object of the present invention to provide a method of preparing such compositions or materials whereby the DOPA and/or catecholic functionality is preserved.

It can also be an object of the present invention to provide one or more adhesive polymeric systems which can be tailored by way of composition, molecular weight and/or concentration to provide desired gelation properties.

It can also be an object of the present invention to provide a synthetic strategy and structural design for the surface modification of a wide range of material substrates. It can also be an object of the present invention to provide a composition and/or related method for facile and efficient modification of a substrate surface, such modification without resort to expensive equipment or complex synthetic procedures.

Other objects, features, benefits and advantages of the present invention will be apparent from this summary and its descriptions of various preferred embodiments, and will be readily apparent to those skilled in the art having knowledge of various polymeric architectures or systems, their adhesive properties and/or the production thereof. Such objects, features, benefits and advantages will be apparent from the above as taken in conjunction with the accompanying examples, data, figures and all reasonable inferences to be drawn therefrom.

The present invention provides novel polymeric compositions through incorporation of one or more DOPA moieties- DOPA-containing or catecholic moieties and/or DOPA/catecholic-like moieties or components. Such compositions are available as described, below, and/or through a general synthetic procedure for polymer end-group activation. With respect to the latter, various polymers or monomeric components thereof can preferably be activated using carbonate chemistry. In particular, a succinimidyl carbonate-activated polymeric component reacted with DOPA or a DOPA-derivative can provide a stable urethane conjugate. Illustrating several preferred embodiments, two possible pathways (a) and (b) in Scheme 1, below, show coupling with a poly(alkylene oxide) in either aqueous or non-aqueous solvents, without compromising desired bioadhesion. Oscillating rheometry and differential scanning calorimetry show that, depending upon the polymeric component, such DOPA-modified polymers have the ability to form polymer hydrogels by a thermally triggered self-assembly process. With respect to various other preferred embodiments, DOPA-containing or such structurally-related polymers can be adsorbed from solution or liquid media for purposes of surface modification and/or particulate stabilization. hi part, the present invention is a biomimetic adhesive composition, including 1) a polymeric component providing or having a surface active effect such as described herein, and 2) at least one catecholic component coupled to the polymeric component. Various polymeric components providing surface active effect will be well-known to those skilled in the art made aware of this invention, such surface activity as can relate to reduced particulate agglomeration and anti-biofouling. For instance, the polymeric component can be water soluble, depending upon end use application, and/or capable of micelle formation depending upon various other end use applications. Preferably, the polymeric component is poly(ethylene oxide)(PEO) or poly(ethylene glycol)(PEG), depending upon monomeric starting material and subsequent polymerization, and can further include one or more hydrophobic components, as described below.

Compositionally, distinct from the preceding and/or as can be separately distinguished from the prior art, the present invention can, alternatively, include a biomimetic adhesive composition, such a composition having (1) a polymeric component and (2) at least one catecholic component conjugated and/or coupled to the polymeric component, such a polymeric component providing a surface active effect and including but not limited to a poly(alkylene oxide). Such a polymeric component can be a poly(alkylene oxide) known in the art or a co-polymer thereof. Preferably, the polymeric component includes poly(ethylene oxide), as can be provided therewith as part of a block co-polymer system. Without limitation to any one poly(alkylene oxide), such a polymeric component can be structurally modified, as described elsewhere herein, with regard to polymer composition, catecholic component and/or the coupling or conjugation therewith.

Regardless, the catecholic component of the present invention is preferably a DOPA precursor, structure, moiety and/or residue conjugated to the polymeric component, such a precursor, residue or moiety as can be incorporated into a peptide or oligopeptide component conjugated with the polymeric component. Such a residue can be derivatized, as would be understood by those skilled in the art, such derivitization limited only by the compositional retention of some adhesive characteristic. Likewise, the catecholic moiety of such a component can be structurally modified or functionally protected insofar as adhesive characteristics are retained or available with subsequent synthetic manipulation. The catecholic component can be coupled to the polymeric component through a variety of synthetic procedures as would be understood by those skilled in the art or as otherwise described herein, depending upon end group functionality. For instance, a DOPA residue can be coupled to a polymeric component to provide the desired conjugate composition, through either urethane or amide bond formation.

More particularly, if coupled to the polymeric component via urethane bond formation, the carboxylic acid group of the DOPA component can be esterified or derivatized with various other functional groups. Alternatively, the DOPA component can be coupled to a polymeric component (e.g., amidation or esterification depending on polymer end group, —NH₂ or —OH) providing a DOPA functionality which can be derivatized by any of numerous known protecting groups, including without limitation the boc protecting group. Conversely, N-group protection of a DOPA component can leave the carboxylic acid group available for multi-functional derivatization and/or a higher density of polymeric components conjugated therewith. Retention of catecholic functionality and/or a related dihydroxy structural relationship can be illustrated using a dopamine component, whereby conjugation with a suitable polymeric component can be achieved with one of the several coupling strategies described herein.

Accordingly, in part, the present invention is an adhesive polymeric composition including at least one of a DOPA residue or a DOPA-derived residue, such residue having a catecholic moiety, and further including at least one monomer coupled to the amino nitrogen of the DOPA residue. Various polymers can be incorporated into such a composition, including without limitation any poly(alkylene oxide), whether commercially-available or as can be prepared via synthetic procedures well known to those skilled in the art. Such polymers can be viewed or considered as derived from the corresponding monomer, as is consistent with the poly(alkylene oxide) nomenclature and acronym (PAO) used herein—although other nomenclature schemes can be used for reference purposes. Preferred embodiments include poly(alkylene oxide) block copolymers such as those available under the Pluronic® trade name/mark.

Consistent with the broader aspects of this invention, as would be understood by those skilled in the art made aware thereof, other polymeric and/or copolymeric components can be used in conjunction with the inventive compositions. More particularly with respect to polymeric hydrogels, hydrophilic and/or hydrophobic blocks can be provided through other copolymeric components. For instance, a hydrophilic block of poly(ethlyene glycol) can be used in conjunction with degradable, hydrophobic blocks such as poly(lactic acid), poly(glycolic acid) and poly(caprolactone) or other degradable polyesters. Alternatively, as a further embodiment of this invention, a random copolymer, such as poly (lactic-co-glycolic acid) can be used as a hydrophobic block.

Relating more particularly to surface modification, preferred polymeric components include PEG and derivatives thereof over a molecular weight range, such a molecular weight as desired for a particular end use application and/or such that the resulting composition is soluble in either a chosen aqueous or organic solvent system. For instance, other polymers useful for preparation of anti-fouling surfaces and particle stabilization include but are not limited to polymers of hyaluronic acid and dextrans, such polymers as can further include incorporation of hydrophobic copolymeric components such as PPO. Various other embodiments of such compositions can be as illustrated by way of several examples, below.

As mentioned above, compositions of this invention useful for purposes of surface modification can include at least one of a DOPA residue or a DOPA-derived residue/component coupled to a particular polymeric component the structure of which can correspond to a desired end-use application. As illustrated in several of the following examples, a preferred DOPA component is a DOPA residue. Other useful components can include, without limitation, DOPA-containing peptides and oligopeptides, whether natural or synthetic in origin. For instance, the consensus decapeptide repeat sequence/mussel adhesive protein (MAP) of the blue mussel Mytilus edulis illustrates one such alternative. Various other DOPA-related bioadhesive components can be used as described elsewhere herein, such components preferably incorporated into the present compositions preserving catecholic functionality and/or related bioadhesive function.

With regard to surface modification, the present invention also includes, in part, a method of using a catecholic and/or DOPA component to incorporate or adsorb a polymeric composition onto a substrate surface. Such a method includes 1) providing a solution or liquid medium of a biomimetic adhesive composition of the type described above, having a polymeric component and a catecholic/DOPA component; and 2) contacting the substrate with the solution/medium to incorporate or facilitate adsorption of the composition on the surface thereof. In various preferred embodiments, a suitable substrate has a surface area, on which incorporation of the inventive composition can provide anti-biofouling properties. In various other preferred embodiments, such a substrate is a particulate, stabilization of which in a fluid medium is imparted by such incorporation. With regard to the latter, various particulate substrates can be produced or prepared within the aforementioned solution such that the polymeric composition is incorporated thereon upon particulate formation. Regardless, a wide range of substrate and/or particulate materials can be used therewith, including, but not limited to, glass, metals, metal oxides and semiconductor compositions. Accordingly, the present invention also includes a corresponding range of composite materials, including such a substrate/particulate and a polymeric composition thereon.

As can be gathered from the preceding, various polymeric compositions of this invention can be designed and prepared to provide various micellization and/or thermal gelation properties. Alternatively, or in conjunction therewith, degradation into excretable polymer components and metabolites can be achieved using, for instance, polyethylene glycol and lactic/glycolic acids, respectively. Regardless, the polymeric compositions of this invention provide improved adhesion by incorporation of one or more DOPA and/or DOPA-derived residues, such incorporation resulting from the coupling of a terminal monomer of the polymeric component to such a residue. Without limitation to any particular synthetic scheme or method of preparation, preferred compositions of this invention can include but are not limited to a urethane moiety between each such terminal monomer and DOPA residue. As described more fully below, such a moiety is a synthetic artifact of the agent/reagent utilized to couple the DOPA residue with the polymeric component. Within the broader aspects of this invention, various other moieties are contemplated, as would be understood by those skilled in the art made aware of this invention, depending upon terminal monomer functionality and choice of coupling agent.

Accordingly, in part, the present invention is also a method of using urethane synthesis to incorporate a DOPA residue into a polymeric system. Such a method includes (1) providing a polymeric component terminating in a plurality of monomers, each having a functional end group; (2) preparing a carbonate derivative of the polymeric component; and (3) preparing a urethane moiety upon reaction of the carbonate derivative and at least one of a DOPA and a DOPA-derivative. As described above, a polymeric component utilized in conjunction with this method can include those having terminal monomeric functionality reactive with a reagent providing the desired carbonate derivative and, ultimately, providing a urethane moiety coupling the polymeric and DOPA components. In preferred embodiments, a preferred coupling reagent is succinimidyl carbonate, described more fully below, and reactive with hydroxy-terminating polymeric components. Various other coupling reagents and/or hydroxy-terminating polymeric components can be used to provide the desired urethane moiety. Without limitation, a preferred embodiment of this inventive method is the use of DOPA or a DOPA-derived component to enhance the adhesive properties of a poly(alkylene oxide). In such preferred embodiments, the polymeric component is selected from one of several commercially available block copolymers. However, as understood from the preceding and the following discussion, various other polymeric components can be utilized to achieve desired physical or functional properties.

In part, the present invention is also a method of using a carbonate intermediate to maintain catecholic functionality of a DOPA-incorporated polymeric composition and/or system, or to otherwise enhance the adhesion properties thereof. Such a method includes (1) providing a polymeric component terminating in a plurality of monomers each having a functional end group; (2) reacting the polymeric component with a reagent to provide a carbonate intermediate; and (3) reacting the carbonate intermediate with at least one of DOPA or a DOPA-derivative. Without limitation to any one theory or mode of operation, this inventive method can be considered by way of enhancing the reactivity of the polymeric component end group, via a suitable carbonate intermediate. Subsequent reaction at the amino-nitrogen of DOPA or a DOPA derivative provides the corresponding conjugate while maintaining catecholic functionality.

In part, the present invention is also a method for the non-oxidative gelation of a DOPA-incorporated polymeric composition and/or system. Such a method includes (1) providing a DOPA-incorporated polymeric composition, including but not limited to, a composition selected from those described above, such a composition having a DOPA or DOPA-derived residue with a substantial catecholic functionality; (2) admixing water and said polymeric composition; and (3) increasing admixture temperature sufficient to gel the polymeric composition, such temperature increase without oxidation of the polymer or DOPA residue incorporated therein. As described more fully below, depending upon choice and identity of the polymeric component of such a composition, an increase in admixture concentration can reduce the temperature required to effect gelation. Related thereto, depending upon choice and identity of a particular copolymeric component, a larger hydrophilic block thereof can increase the temperature required to gel the corresponding composition. As described more fully below, various other structural and/or physical parameters can be modified to tailor gelation, such modifications as can be extended to other polymeric compositions and/or systems—consistent with the broader aspects of this invention.

Succinimidyl carbonate has previously been recognized as a useful reagent for activating hydroxyl groups of small organic compounds and PEG molecules to form urethane derivatives for biomolecular binding.^{38,39} With respect to various preferred embodiments, succinimidyl carbonate was used for the first time with the present invention to activate the hydroxyl groups of commercially-available poly(alkylene oxides) in the presence of 4-(dimethylamino)pyridine (DMAP). The resulting intermediates can be stably stored as solids in a desiccator at 20° C. and have been found to maintain their activity after several months of storage.

In accordance with this invention, as demonstrated in Scheme 1, various synthetic routes can be used to couple DOPA moieties to such carbonate activated intermediates. DOPA methyl ester (DME), prepared by the reaction of DOPA with methanol in the presence of thionyl chloride,³⁴ can be used in organic solvents. Reaction progress can be monitored by TLC and NMR, with the coupling reaction virtually complete in one hour (with representative conjugates DME-PAO7 and DME-PAO8). High product yields were obtained upon purification from cold methanol.

The free carboxylic form of DOPA can be coupled with the carbonate intermediate in alkaline aqueous solution. There have been several reports studying the introduction of DOPA into peptides in solid- and liquid-phase chemistry.^{29,40-43} It is well-known that the chief difficulty in working with DOPA is its ease of oxidation (to DOPA-quinone and other products), which readily occurs in alkaline aqueous solutions.^{40,41} To prevent unwanted oxidation of DOPA catechol side chains during coupling under alkaline conditions, a borate-protected DOPA can be first formed by adding DOPA to aqueous sodium borate (Scheme 1). The resulting complex is remarkably stable in neutral or alkaline solutions,⁴¹ and can be readily deprotected under acidic conditions. Taking advantage of complexation between DOPA and borate, DOPA was coupled to the ends of several commercially-available PAOs under alkaline aqueous conditions to yield DOPA-PAO7 and DOPA-PAO8. Visual inspection of the reaction solution revealed the absence of strongly absorbing DOPA-quinone, an indication that DOPA remains unoxidized during the reaction. At the completion of the reaction, acidification with HCI resulted in deprotection of the DOPA endgroups of the block copolymer.

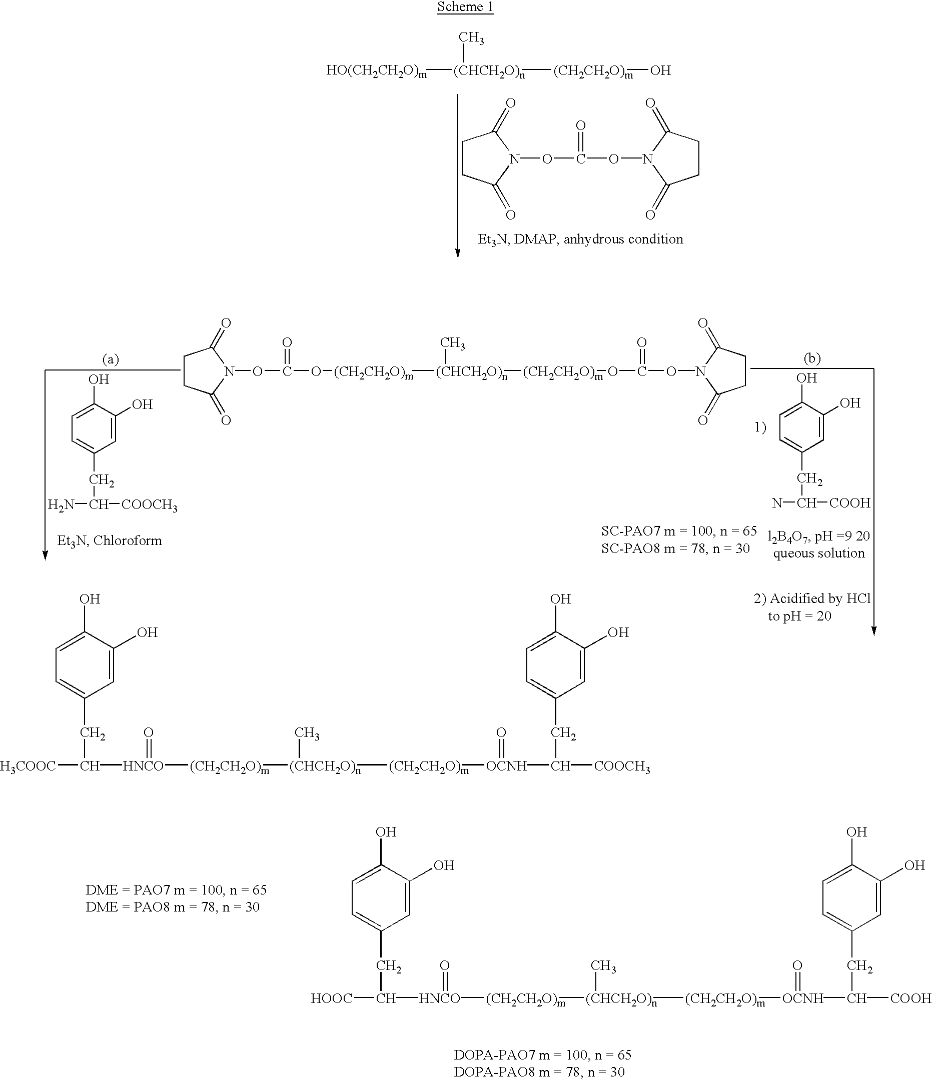

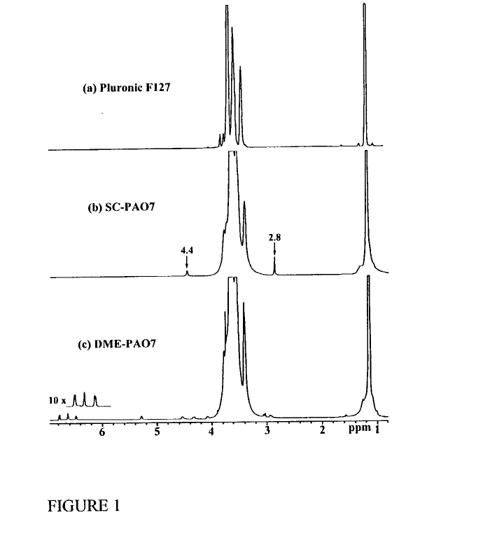

Both ¹H NMR spectra and colorimetric assay confirmed the compositions of the succinimidyl activated reaction intermediates and all four DOPA-modified PAOs of Scheme 1. Shown in FIG. 1 are ¹H NMR spectra of PAO Pluronic® F127, the succinimidyl carbonate activated intermediate (SC-PAO7), and the corresponding DOPA methyl ester modified PAO (using Pluronic® F127, DME-PAO7). The sharp peaks at ˜2.8 ppm due to the —CH₂— rotons from the succinimidyl carbonate group and at ˜4.4 ppm due to the —CH₂—O— protons from the only ethylene oxide group adjacent to the carbonate group in activated-PAO completely disappear from the ¹H NMR spectra of the DOPA-containing PAO, whereas a series of new peaks appear due to the introduction of DOPA moieties into the copolymers. One characteristic feature of the ¹H NMR spectra of the DOPA-containing PAO is the appearance of one singlet and two doublets in the range of 6.5-6.9 ppm corresponding to the three protons on the DOPA phenyl ring. Similar features were also observed in the ¹H NMR spectrum (not shown) of the DOPA-PAO conjugate synthesized from aqueous solution. Based on the assumption of two available succinimidyl carbonate groups in the corresponding carbonate intermediates, SC-PAO7 and SC-PAO8, coupling efficiencies of DOPA methyl ester and DOPA to these two PAOs were quantitatively found to be in the range from 76% to 81% as obtained from colorimetric analysis (Table 1). The reported coupling efficiencies are the average values of at least three repeated syntheses performed under the same conditions and were not found to increase significantly when a larger excess of DOPA was used in the reaction. Similar coupling efficiencies were also found for DOPA-PAO7 (from PAO Pluronic® F127) and DOPA-PAO8 (from PAO Pluronic® F68) made from aqueous solutions, suggesting that the hydrolysis of succinimidyl carbonate activated PAOs is slow in the aqueous alkaline solution containing Na₂B₄O₇.

In contrast to coupling efficiencies, the product yields (shown in Table 1) of selected DOPA-modified PAOs synthesized in aqueous solution were found to be lower than those synthesized in organic solvent. This may be due to the surfactant properties of the starting PAO material, causing the low efficiency of extraction of DOPA-modified PAO with dichloromethane from water. It should be noted that the free carboxylic acid in DOPA-PAO7 and DOPA-PAO8 can be further functionalized using standard peptide chemistry to tailor the properties of the block copolymers. As referenced above, the four DOPA-modified PAOs of Table 1 could be stored at −20° C. indefinitely with no discoloration or change in properties.

TABLE 1
Coupling efficiency and product yield of DOPA modified Pluronic ®.

| | Coupling Efficiency (%)* | Product Yield (%) |
|---|---|---|
| DME-PAO7 | 78.0 ± 4.0 | 75.0 ± 5.0 |
| DOPA-PAO7 | 80.0 ± 4.0 | 52.0 ± 3.0 |
| DME-PAO8 | 76.0 ± 2.0 | 76.0 ± 4.0 |
| DOPA-PAO8 | 81.0 ± 2.0 | 49.0 ± 2.0 |

*Determined by colorimetric analysis.³⁰

It is widely acknowledged that the commercially-available Pluronic® block copolymers self-assemble in a concentration- and temperature-dependent manner into micelles consisting of a hydrophobic PPO core and a water-swollen corona consisting of PEO segments.^{14,15,44-47} At high concentration, certain PEO-PPO-PEO block copolymers, such as Pluronic® F 127 and Pluronic® F68, transform from a low viscosity solution to a clear thermoreversible gel at elevated temperature. It is generally assumed¹⁴ that the interactions between micelles at elevated temperature lead to the formation of a gel phase, which is stabilized by micelle entanglements. The micellization and gelation processes have been found to depend on factors such as block copolymer molecular weight, relative block sizes, solvent composition, polymer concentration, and temperature.^{14,47,48} For example, increasing the length of the hydrophilic PEO blocks relative to the hydrophobic PPO block results in an increase in micellization and gelation temperature (T_{gel}).⁴⁹

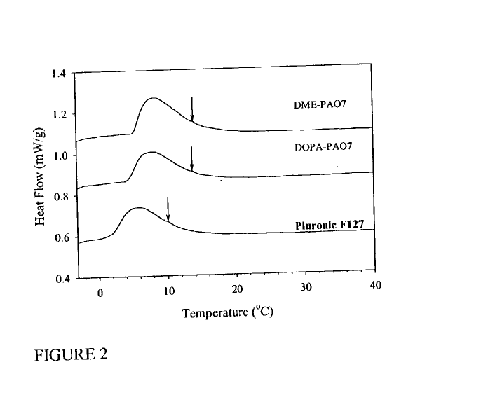

Differential scanning calorimetry (DSC) measurements were performed on aqueous solutions of DME-PAO7 and DOPA-PAO7 at different concentrations to detect aggregation of block copolymers into micelles. DSC profiles obtained for Pluronic® F127, DME-PAO7 and DOPA-PAO7 were found to be qualitatively similar and were characterized by a large endothermic transition corresponding to micelle formation followed by a small endotherm at T_{gel} ( FIG. 2). The transition temperature of the small peak was found to correlate strongly with T_{gel} determined by rheometry and the vial inversion method (Table 2).

TABLE 2
Gel temperatures obtained from vial inversion method, rheology or
differential scanning calorimetry for 22 wt % DME-PAO7,
DOPA-PAO7 and Pluronic ® F127 solutions.

| | Gel Temperature (° C.) | | |
|---|---|---|---|
| | vial inversion | | |
| | method | Rheological | DSC |
| DME-PAO7 (22 wt %) | 22.0 ± 1.0 | 20.3 ± 0.6 | 20.9 ± 0.1 |
| DOPA-PAO7 (22 wt %) | 22.0 ± 1.0 | 20.4 ± 0.5 | 21.7 ± 0.2 |
| Pluronic ® F127 (22 wt %) | 17.0 ± 1.0 | 15.4 ± 0.4 | 17.5 ± 0.4 |

Aqueous solutions with concentrations ranging from 10 to 30% (w/w) of DOPA-PAO7 copolymers and 35 to 54% (w/w) of DOPA-PAO8 copolymers were prepared by the cold method,⁵⁰ in which DOPA conjugate was dissolved in distilled water at ca. 4° C. with intermittent agitation until a clear solution was obtained. Thermal gelation of concentrated solutions was initially assessed using the vial inversion method.¹⁵ In this method, the temperature at which the solution no longer flows is taken as the gelation temperature.

The gelation temperature was found to be strongly dependent on copolymer concentration and block copolymer composition (i.e., PAO7 versus PAO8). For example, 22 wt % solutions of DOPA-PAO7 and DME-PAO8 were found to form a transparent gel at approximately 22.0±1.0° C.; decreasing the polymer concentration to 18 wt % resulted in a gelation temperature of approximately 31.0±1.0° C. However, DOPA-PAO7 solutions with concentrations less than 17 wt % did not form gels when heated to 60° C. DOPA-PAO7 exhibits a slightly higher gel temperature than that (17.0±1.0° C.) of unmodified Pluronic® F127. The gelation behavior of DOPA-PAO8 was found to be qualitatively similar, except that much higher polymer concentrations were required to form a gel. 54 wt % solutions of DOPA-PAO8 and DME-PAO8 formed gels at 23.0±1.0° C., while 50 wt % of DOPA-PAO8 gels at 33.0±1.0° C. However, DOPA-PAO8 solutions with concentrations less than 35 wt % did not form gels when heated to 60° C. DOPA-PAO8 exhibits a much higher gel temperature than that (16.0±1.0° C.) of unmodified Pluronic® F68. These gels were found to be resistant to flow over long periods of time. From this experiment, we have also found that both DOPA and DOPA methyl ester-derivatives of the same commercially available Pluronic® PAO exhibit almost the same gel temperature, and the gel made from 54 wt % of either DME-PAO8 or DOPA-PAO8 at room temperature is stiffer than that made from 22 wt % of either DME-PAO7 or DOPA-PAO7.

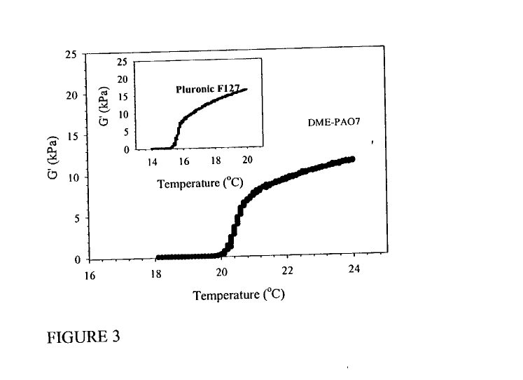

The viscoelastic behavior of DOPA-modified Pluronic® solutions was further studied by oscillatory rheometry. FIG. 3 shows the elastic storage modulus, G′, of 22 wt % solutions of unmodified Pluronic® F127 and DME-PAO7 aqueous solutions as a function of temperature. Below the gelation temperature, storage modulus G′ was negligible, however G′ increased rapidly at the gel temperature (T_{gel}), defined as the onset of the increase of the G′ vs. Temperature plot.⁵¹ DOPA-PAO7 (not shown) exhibited a similar rheological profile. The T_{gel} of 22 wt % solutions of DME-PAO7 and DOPA-PAO7 were found to be identical

(20.3±0.6° C.), which is approximately 5 degrees higher than an equivalent concentration of unmodified- Pluronic® F127 (15.4±0.4° C.). G′ of DME-PAO7 or DOPA-PAO7 approaches a plateau value of 13 kPa, which is comparable to that of unmodified Pluronic® F127 and in agreement with the reported results.52

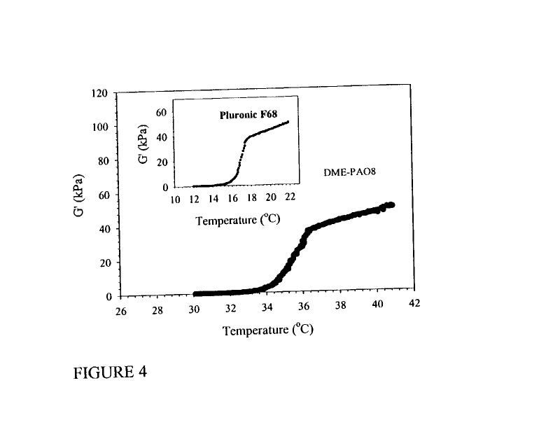

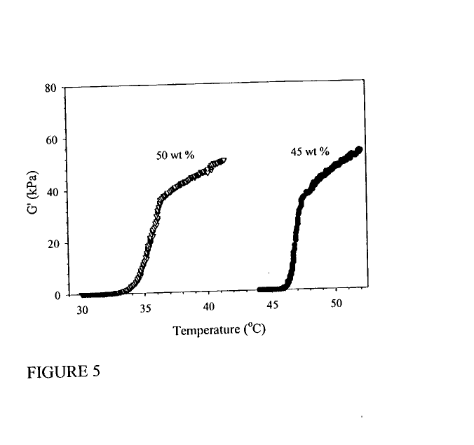

Shown in FIG. 4 are the rheological profiles of 50 wt % solutions of unmodified Pluronic® F68 and DME-PAO8 as a function of temperature. The T_{gel} of a 50 wt % DME-PAO8 solution was found to be 34.1±0.6° C., whereas the T_{gel} of an equivalent concentration of unmodified Pluronic® F68 was approximately 18° C. lower (16.2±0.8° C.). The plateau storage moduli of 50 wt % solutions of DME-PAO8 and unmodified Pluronic® F68 were not significantly different, approaching a plateau value as high as 50 kPa. The concentration dependence of T_{gel} is illustrated in FIG. 5, which shows the rheological profile of DME-PAO8 at two different concentrations as a function of temperature. T_{gel} of 45 wt % solution of DME-PAO8 was observed to be approximately 12° C. higher than that of 50 wt % solution of DME-PAO8, which is in agreement with the trend of increasing T_{gel} with decreasing concentration as reported in the literature.⁵²

Since both DOPA and DOPA methyl ester can be considered hydrophilic, the increase of T_{gel} observed in the DOPA-modified Pluronic® PAOs, compared with that of unmodified Pluronic® PAOs, is likely due to the increase in length of the hydrophilic PEO segments resulting from coupling of DOPA to the endgroups. It is clear from the data shown in FIGS. 3 and 4 that the coupling of DOPA or DOPA methyl ester to the Pluronic® PAO endgroups has a more significant impact on the T_{gel} of Pluronic® F68 compared to Pluronic® F127. This can be rationalized in terms of the overall molecular weights of F68 (approx. 8,600) and F127 (approx. 12,600). Addition of DOPA and DOPA methyl ester to both endgroups using the chemistry shown in Scheme 1 results in an increase in molecular weight of 446 and 474, respectively. This represents a larger % molecular weight increase for F68 compared to F127, due to lower base molecular weight of F68.

The data presented herein is in agreement with previous calorimetry studies of unmodified Pluronic® PAOs, which demonstrated that the broad peak at low temperature is due to micellization while the small peak at higher temperature, only observed in concentrated solutions, corresponds to gelation, a nearly athermal process. As seen in Table 3, the onset temperature of micellization, the temperature at maximum heat capacity and T_{gel} of unmodified Pluronic® F127 were found to be lower than those of DOPA-PAO7, whereas the specific enthalpies determined from the areas under the transition (FIG. 2) are approximately the same. These enthalpies include contributions from both micellization and gelation. However, due to the small enthalpy of gelation, the observed enthalpy changes can be largely attributed to micellization.

TABLE 3
Comparisons of 30 wt % DME-PAO7, DOPA-PAO7 and unmodified
Pluronic ® F127 solutions on onset micellization
temperature, temperature at maximum heat capacity,
enthalpies, and gel temperature from differential
scanning calorimetry experiments.

| | | Temperature at | | |
|---|---|---|---|---|
| | Micellization | Maximum heat | | Gel |
| | temperature | capacity | ΔH | Temperature |
| | (° C.) | (° C.) | (J/g) | (° C.) |
| DME-PAO7 | 5.2 ± 0.2 | 8.3 ± 0.1 | 20.3 ± 2.4 | 14.0 ± 0.4 |
| (30 wt %) | | | | |
| DOPA-PAO7 | 4.6 ± 0.2 | 8.0 ± 0.6 | 19.3 ± 1.4 | 14.0 ± 0.2 |
| (30 wt %) | | | | |
| Pluronic ® | 1.9 ± 0.3 | 6.0 ± 0.4 | 20.6 ± 1.6 | 10.6 ± 0.6 |
| F127 (30 wt %) | | | | |

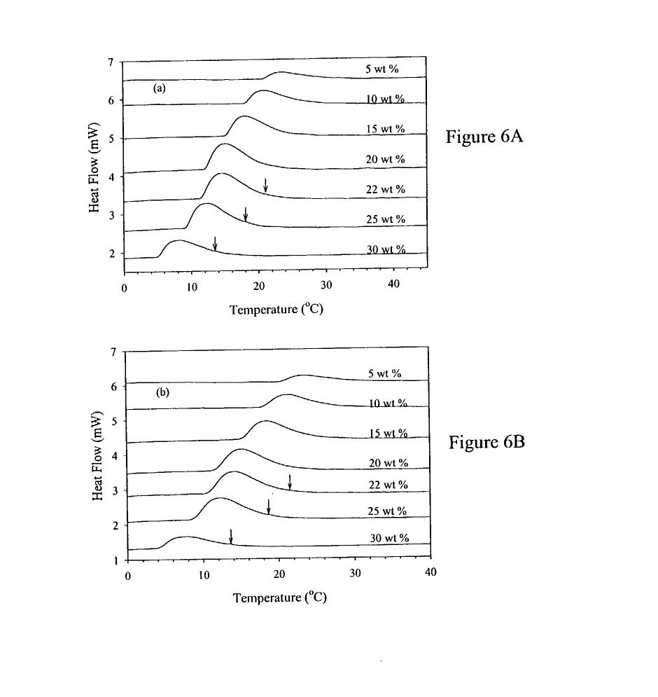

The micellization peak was seen to extend to temperatures above the onset of gelation, indicating that additional monomers aggregate into micelles at temperatures above the gelation point.^{14,46} The concentration dependence of DOPA-PAO7 and DME-PAO7 aggregation is shown in FIG. 6. DSC thermograms indicate a decrease in micellization temperature and T_{gel} with increasing polymer concentration. The broad endothermic peak corresponding to micellization can also be observed in solutions at concentrations at which no gelation takes place; the characteristic temperature of the broad peak increases linearly with decreasing copolymer concentration, whereas the small peak was observed to coincide to the gel temperature of the concentrate copolymers but disappears as copolymer concentration decreases.

### EXAMPLES OF THE INVENTION

The following non-limiting examples and data illustrate various aspects and features relating to the compositions and/or methods of the present invention, including the production of various polymeric or co-polymeric compositions having incorporated therein one or more DOPA or DOPA-derived components, as are available through the synthetic methodology described herein. In comparison with the prior art, the present compositions and methods provide results and data which are surprising, unexpected and contrary to the prior art. While the utility of this invention is illustrated through the use of several polymeric or co-polymeric systems, it will be understood by those skilled in the art that comparable results are obtainable with various other compositions and/or methods for preparation, as are commensurate with the scope of this invention.

PEO₁₀₀PPO₆₅PEO₁₀₀ (Pluronic® F127, Mw=12,600) and PEO₇₈PPO₃₀PEO₇₈

(Pluronic® F68, Mw=8,400) were purchased from Sigma (St. Louis, Mo.). L-DOPA, thionyl chloride, N,N-disuccinimidyl carbonate, sodium borate, sodium molybdate dihydrate, sodium nitrite as well as 4-(dimethylamino)pyridine (DMAP) were purchased from Aldrich (Milwaukee, Wis.). Acetone was dried over 4A molecular sieve and distilled over P₂O₅ prior to use. Triethylamine was freshly distilled prior to use. All other chemical reagents were used as received. L-DOPA methyl ester hydrochloride was prepared according to literature procedures.³⁴

Glass coverslips (12 mm dia.) used in the following examples were cleaned by immersing in 5% Contrad 70 solution (Decon Labs, Inc.) in an ultrasonic bath for 20 minutes, rinsed with DI H₂O, sonicated in DI H₂O for 20 minutes, rinsed in acetone, sonicated in acetone for 20 minutes, rinsed in hexanes, sonicated in hexanes for 20 minutes, rinsed in acetone, sonicated in acetone for 20 minutes, rinsed in DI H₂O, and sonicated in DI H₂O for 20 minutes. The coverslips were subsequently air-dried in a HEPA-filtered laminar flow hood. To create pristine gold substrates, clean coverslips were sputtered (Cressington 208HR) with 2 nm Cr followed by 10 nm Au (99.9% pure).

Both pristine and modified gold surfaces were characterized, as described below, by X-ray photoelectron spectroscopy (XPS). XPS data was collected on an Omicron ESCALAB (Omicron, Taunusstein, Germany) configured with a monochromated AIKα (1486.8 eV) 300-W X-ray source, 1.5 mm circular spot size, a flood gun to counter charging effects, and an ultrahigh vacuum (<10⁻⁸ Torr). The takeoff angle, defined as the angle between the substrate normal and the detector, was fixed at 45°. Substrates were mounted on standard sample studs by means of double-sided adhesive tapes. All binding energies were calibrated using either the Au(4ƒ_{7/2}) gold peak (84.0 eV) or the C(1s) carbon peak (284.6 eV). Analysis consisted of a broad survey scan (50.0 eV pass energy) and a 10-minute high-resolution scan (22.0 eV pass energy) at 270-300 eV for C(1s). Peak deconvolution and atomic percent calculations were performed with EIS analysis software.

Secondary ion spectra were collected on a TRIFT III™ time-of flight secondary ion mass spectrometer (TOF-SIMS) (Physical Electronics, Eden Prairie, Minn.) in the mass range 0-2000 m/z. A Ga⁺-source was used at a beam energy of 15 keV with a 100 μm raster size. Both positive and negative spectra were collected and calibrated with a single set of low mass ions using the PHI software Cadence.

To determine relative hydrophilic/hydrophobic nature of the surfaces, contact angle data was collected, as described below, by the sessile drop method. A custom-built contact angle goniometer (components from Ramé-Hart, Mountain Lakes, N.J.) equipped with a humidified sample chamber was used to measure both advancing and receding contact angles of ultrapure water (18.2MΩ-cm; Barnstead, Dubuque, Iowa) on unmodified and modified substrates. For each surface, four measurements were made at different locations and the mean and standard deviation were reported.

Surface Plasmon Resonance (SPR) measurements were made on a BIACORE 2000 (Biacore International AB; Uppsala, Sweden) using bare gold sensor cartridges. The resonance response was calibrated using 0-100 mg/ml NaCl solutions. Dilute solutions (0.1 mM in H₂O) of mPEG-DOPA, mPEG-MAPd, and mPEG-OH were injected into the SPR flow cell for 10 min after which flow was switched back to pure DI H₂O. In a separate experiment to measure protein adsorption to modified substrates, sensor surfaces with preformed PEG films were exposed to 0.11 mg/ml bovine serum albumin (BSA) solution in 10 mM HEPES buffer (0.15M NaCl, pH=7.2), and subsequently pure buffer.

For use in demonstration of anti-fouling effects, NIH 3T3-Swiss albino fibroblasts obtained from ATCC (Manassas, Va.) were maintained at 37° C. and 10% CO₂ in Dulbecco's modified Eagle's medium (DMEM; Cellgro, Hemdon, Va.) containing 10% (v/v) fetal bovine serum (FBS) and 100U/ml of both penicillin and streptomycin.

With regard to the following cell adhesion tests and/or spreading assays, modified and unmodified substrates were pretreated in 12-well TCPS plates with 1.0 ml of DMEM containing 10% FBS for 30 minutes at 37° C. and 10% CO₂. Fibroblasts of passage 12-16 were harvested using 0.25% trypsin-EDTA, resuspended in DMEM with 10% FBS, and counted using a hemocytometer. Cells were seeded at a density of 2.9×10³ cell/cm² by diluting the suspension to the appropriate volume and adding 1 ml to each well. The substrates were maintained in DMEM with 10% FBS at 37° C. and 10% CO₂ for 4 hours, after which time unattached cells were aspirated. Adherent cells on the substrates were fixed in 3.7% paraformaldehyde for 5 min and subsequently treated with 5μM 1,1′-dioctadecyl-3,3,3′,3′-tetramethylindocarbocyanine perchlorate (DiI; Molecular Probes, Eugene, Oreg.) in DMSO for 30 minutes at 37° C. The stain was then aspirated and substrates were washed (3×) with DMSO for 10 minutes and mounted on glass slides using Cytoseal (Stephens Scientific, Kalamazoo, Mich.) to preserve fluorescence. These experiments were performed in triplicate for statistical purposes. For electron microscopy, some samples were dehydrated with EtOH after fixing, critical-point dried, and sputtered with 3 nm Au.

To quantify cell attachment, substrates were examined with an Olympus BX-40 (λ_{Ex}=549 nm, X_{Em}=565 nm) and color images were captured with a Coolsnap CCD camera (Roper Scientific, Trenton, N.J.). Five images were taken from each of the three substrate-replicates. The resulting images were quantified using thresholding in Metamorph (Universal Imaging, Downington, Pa.). A one-way ANOVA and Tukey's post-hoc test with 95% confidence intervals (SPSS, Chicago, Ill.) were used to determine statistical significance of the data. The mean and standard deviation of the measurements were reported.

### Example 1

### Synthesis of Succinimidyl Carbonate PAO, SC-PAO7

Pluronic® F127 (0.60 mmols) was dissolved in 30 mL of dry dioxane. N,N′-Disuccinimidyl carbonate (6.0 mmols) in 10 mL dry acetone was added. DMAP (6.0 mmols) was dissolved in 10 mL dry acetone and added slowly under magnetic stirring. Activation proceeded 6 hours at room temperature, after which SC-PAO7 was precipitated into ether. The disappearance of the starting materials during the reaction was followed by TLC in chloroform-methanol (5:1) solvent system. The product was purified by dissolution in acetone and precipitation with ether four times. The product yield was 65%. ¹H NMR (500 MHz, CDCl₃): δ ppm 0.96-1.68 (br, —OCHCH₃CH₂O—), 2.80 (s, —COON(CO)₂(CH₂)₂), 3.15-4.01 (br, —OCH₂CH₂O—; —OCHCH₃CH₂O—), 4.40 (s, —OCH₂CH₂OCOON(CO)₂CH₂—).

### Example 2

### Synthesis of DME-PAO7

A slurry of DOPA methyl ester hydrochloride (1.25 mmols) and triethylamine

(2.5 mmols) was mixed with SC-PAO7 (0.16 mmols) in 10 mL chloroform. The disappearance of the starting materials during the reaction was followed by TLC in chloroform-methanol-acetic acid (5:3:1) solvent system. After stirring for 1 hour at room temperature, the solvent was evaporated off, and DME-PAO7 was purified by precipitation from cold methanol three times. DME-PAO7 gave a positive Arnow test indicating the presence of catechol hydroxyl groups.³⁵ The product yield was 75%. ¹H NMR (500 MHz, CDCl₃): δ ppm 0.98-1.71 (br, —OCHCH₃CH₂O—), 2.83-3.06 (m, —NHCHCH₂C₆H₃(OH)₂COOCH₃), 3.15-4.02 (br, —OCH₂CH₂O—; —OCHCH₃CH₂O—; —NHCH(CH₂C₆H₃(OH)₂COOCH₃), 4.05-4.35 (d, —OCH₂CH₂OCONHCHCH₂C₆H₃(OH)₂COOCH₃), 4.55 (br, —NHCHCH₂C₆H₃(OH)₂COOCH₃), 5.30 (d, —NHCHCH₂C₆H₃(OH)₂COOCH₃), 6.45-6.80 (1s, 2d, —NHCHCH₂C₆H₃(OH)₂COOCH₃).

### Example 3

### Synthesis of DOPA-PAO7

L-DOPA (1.56 mmols) was added to 30 mL 0.1 M Na₂B₄O₇ (pH =9.32) aqueous solution under Ar atmosphere, followed by stirring at room temperature for 30 minutes. SC-PAO7 (0.156 mmols) in 5 mL acetone was added to the resulting mixture and stirred overnight at room temperature. The solution pH was maintained with sodium carbonate during the reaction. The disappearance of the starting materials during the reaction was followed by TLC in chloroform-methanol-acetic acid (5:3:1) solvent system. The solution was acidified to pH 2 with concentrated hydrochloric acid and then extracted three times with dichloromethane. The combined dichloromethane extracts were dried with anhydrous sodium sulfate and filtered, and dichloromethane was evaporated. The product was further purified by precipitation from cold methanol. DOPA-PAO7 gave a positive Arnow test indicating the presence of catechol hydroxyl groups.³⁵ The product yield was 52%. ¹H NMR (500 MHz, CDCl₃): δ ppm 0.92-1.70 (br, —OCHCH₃CH₂O—), 2.91-3.15 (m, —NHCHCH₂C₆H₃(OH)₂COOCH), 3.20-4.10 (br, —OCH₂CH₂O—; —OCHCH₃CH2O—), 4.1-4.35 (d, —OCH₂CH₂OCONHCHCH₂C₆H₃(OH)₂COOH), 4.56 (m, —NHCHCH₂C₆H₃(OH)₂COOH), 5.41 (d, —NHCHCH₂C₆H₅(OH)₂COOH), 6.60-6.82 (1s, 2d, —NHCHCH₂C₆H₃(OH)₂(COOH).

### Example 4

### Synthesis of Succinimidyl Carbonate PAO8, SC-PAO8

A procedure similar to that described above for the synthesis and purification of SC-PAO7 was used to prepare SC-PAO8. The product yield was 68%. ¹H NMR (500 MHz, CDCl₃): δ ppm 0.95-1.58 (br, —OCHCH₃CH₂O—), 2.80 (s, —COON(CO)₂(CH₂)₂), 3.10-4.03 (br, —OCH₂CH₂O—; —OCHCH₃CH₂O—), 4.40 (s, —OCH₂CH₂OCOON(CO)₂CH₂CH₂).

### Example 5

### Synthesis of DME-PAO8

A procedure similar to that described above for the synthesis and purification of DME-PAO7 conjugate was used to make DME-PAO8. The product yield was 76%. ¹H NMR (500 MHz, CDCl₃): δ ppm 0.98-1.50 (br, —OCHCH₃CH₂O—), 2.85-3.10 (m, —NHCHCH₂C₆H₃(OH)₂COOCH₃), 3.15-4.01 (br, —OCH₂CH₂O—; —OCHCH₃CH₂O—; —NHCH(CH₂C₆H₃(OH)₂COOCH₃), 4.03-4.26 (d, —OCH₂CH₂OCONHCHCH₂C₆H₃(OH)₂COOCH₃), 4.55 (m, —NHCHCH₂C₆H₃(OH)₂COOCH₃), 5.30 (d, —NHCHCH₂C₆H₃(OH)₂COOCH₃), 6.45-6.77 (1s, 2d, —NHCHCH₂C₆H₃(OH)₂COOCH₃).

### Example 6

### Synthesis of DOPA-PAO8

A procedure similar to that described above for the synthesis of DOPA-PAO7 conjugate was used to prepare and purify DOPA-PAO8. The product yield was 49%. ¹H NMR (500 MHz, CDCl₃): δ ppm 0.92-1.50 (br, —OCHCH₃CH₂O—), 2.91-3.10 (m, —NHCHCH₂C₆H₃(OH)₂COOH), 3.15-3.95 (br, —OCH₂CH₂O—; —OCHCH₃CH₂O—), 4.06-4.30 (d, —OCH₂CH₂OCO NHCHCH₂C₆H₃(OH)₂COOH), 4.54 (m, —NHCHCH₂C₆H₃(OH)₂COOH), 5.35 (d, —NHCHCH₂C₆H₅(OH)₂COOH), 6.50-6.80 (1s, 2d, —NHCHCH₂C₆H₃(OH)₂COOH).

### Example 7

### Colorimetric Assay

Coupling efficiencies of DOPA methyl ester and DOPA to Pluronics(E) F127 and F68 were determined using a colorimetric method.³⁶ Briefly, samples were analyzed in triplicate by diluting aliquots of standards or unknown solutions with 1 N HCl to a final volume of 0.9 mL. 0.9 mL of nitrite reagent (1.45 M sodium nitrite and 0.41 M sodium molybdate dihydrate) was added to the DOPA solution, followed immediately by the addition of 1.2 mL of 1 N NaOH. Due to time-dependent changes in absorbance intensity, care was taken to ensure that the time between the addition of NaOH and recording of the absorbance was 3 minutes for all standards and samples. The absorbance was recorded at 500 nm for all standards and samples. DOPA was used as the standard for both the DOPA methyl ester and DOPA conjugates.

### Example 8

### Rheology

Rheological measurements of the gelation process were performed using a Bohlin VOR Rheometer (Bohlin Rheologi, Cranbury, N.J.). A 30 mm diameter stainless steel cone and plate geometry with a cone angle of 2.5 degrees was used for all measurements. The temperature was controlled by a circulating water bath. Samples were cooled in the refrigerator prior to transfer of 0.5 mL of liquid solution to the apparatus. Measurements of storage and loss moduli, G′ and G ″, were taken in the oscillatory mode at 0.1 Hz and a strain of 0.45%. The heating rate was 0.5° C./min except in the vicinity of the gelation temperature, when it was reduced to 0.1 ° C./min. The strain amplitude dependence of the viscoelastic data was checked for several samples, and measurements were only performed in the linear range where moduli were independent of strain amplitude. Mineral oil was applied to a ring surrounding the outer surfaces of the sample compartment to prevent dehydration during measurements.

### Example 9

### Differential Scanning Calorimetry (DSC)

DSC measurements were performed on a TA Instruments DSC-2920 (TA Instruments, New Castle, Del.) calorimeter. Spectra were obtained for three samples of each concentration on heating and cooling cycle. Sample volumes of 20 μl in hermetically sealed aluminum pans were used and scans were recorded at a heating and cooling rate of 3° C./min with an empty pan as reference.

### Example 10a

Amino-terminated methoxy-PEG, mPEG-NH₂ (2.0 g, 0.40 mmoles, \overline{M}_{w}=2,000 or 5,000, Sun-Bio PEGShop), N-Boc-L-DOPA dicyclohexylammonium salt (0.80 mmoles), HOBt (1.3 mmoles), and Et₃N (1.3 mmoles) were dissolved in 20 mL of a 50:50 mixture of dichloromethane (DCM) and DMF. HBTU (0.80 mmoles) in 10 mL of DCM was then added, and the reaction was carried out under argon at room temperature for 30 minutes. The reaction solution was successively washed with saturated sodium chloride solution, 5% NaHCO₃, diluted HCl solution, and distilled water. The crude product was concentrated under reduced pressure and purified by column chromatography on Sephadex® LH-20 with methanol as the mobile phase. The product, mPEG-DOPA, was further purified by precipitation in cold methanol three times, dried in vacuum at room temperature, and stored under nitrogen at −20° C. ¹H NMR (500 MHz, CDCl₃/TMS): δ6.81-6.60 (m, 3H, C₆H₃(OH)₂—), 6.01 (br, s, 1H, OH—), 5.32 (br, s, 1H, OH—), 4.22 (br, s, 1H, C₆H₃(OH)₂—CH₂—CH(N—)—C(O)N—), 3.73-3.38 (m, PEO), 3.07 (m, 2H, PEO-CH₂—NH—C(O)—), 2.73 (t, 2H, C₆H₃(OH)₂—CH₂—CH(N—)—C(O)N—), 1.44 (s, 9 H, (CH₃)₃C—), 1.25 (s, 3 H, CH₃CH₂O—).

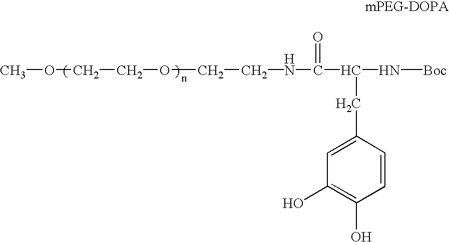

### Example 10b

The synthesis and related procedures of the preceding example can be extended, by analogy, using other DOPA-containing peptides and oligopeptides, whether natural or synthetic in origin. Depending upon a particular synthetic sequence, use of an N-terminal protecting group may be optional. As referenced above, various other DOPA-like or catechol-containing components can also be utilized, as would be well-known to those skilled in the art made aware of this invention. For instance, beta-amino acids and N-substituted glycine DOPA analogs can be used.

Regardless of a particular DOPA or DOPA-like component, a variety of polymeric components can be used in accordance with the synthetic techniques and procedures described above. The polymeric component can vary in molecular weight limited only by corresponding solubility concerns. As mentioned above, a variety of other polymers can be used for surface anti-fouling and/or particle stabilization, such polymers including but not limited to hyaluronic acid, dextrans and the like. Depending upon solubility requirements and desired surface effect, the polymeric component can be branched, hyperbranched or dendrimeric, such components available either commercially or by well-known synthetic techniques.

While the composition of example 10a is the amidation product of the referenced starting materials, it should be understood that comparable polymer-DOPA conjugates can be prepared coupling the N-terminus of a DOPA or DOPA-like component, or a corresponding catecholic component, to an end group, back bone or side chain of a suitably functionalized natural or synthetic polymer, including those described above. For example, and without limitation, as illustrated above, a suitable polymeric component terminating with a carbonate functionality can be used to provide the desired conjugate by reaction with the N-terminus of the desired DOPA, DOPA-like and/or catecholic component.

### Example 11a

The consensus decapeptide repeat sequence(mussel adhesive protein decapeptide, MAPd, NH₂-Ala-Lys-Pro-Ser-Tyr-Hyp-Thr-DOPA-Lys-CO₂H) of the blue mussel Mytilus edulis foot protein 1 (Mefp 1) was synthesized by solid phase peptide synthesis on Rink resin (0.6 mMol/g) using Fmoc protected amino acids, BOP, HOBt, and DIEA as activating agents, and NMP as solvent. Fmoc deprotection was performed using a 25% piperidine solution in NMP for twenty minutes. Couplings of amino acids were performed using two equivalents of the Fmoc-amino acid:BOP:HOBt:DIEA in a 1:1:1:1 ratio for twenty minutes, with an initial, ten-minute preactivation step. Upon completion of the decapeptide, the free amine terminus of the decapeptide was coupled to activated methoxy-PEG-CO₂H (mPEG-SPA, \overline{M}_{w}=2 k or 5 k, Shearwater Polymers) using carbodiimide chemistry. The PEG-decapeptide conjugates (mPEG-MAPd, 2 k or 5 k) were cleaved at 0° C. for two hours using 1 M TMSBr in TFA, with EDT, thioanisole, and m-cresol. The crude mPEG-MAPd products were precipitated in ether at 0° C., and purified by preparative HPLC using a Vydac 218TP reverse phase column (220×22 mm×10 μm). The purity of the products was determined to be >90% using analytical HPLC, and the structures confirmed using a PerSeptive Biosystem MALDI-TOF-MS.

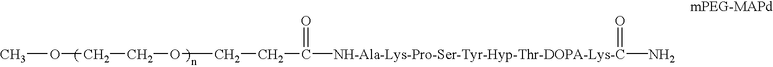

### Example 11b

The synthesis and procedures of example 11 a can be extended analogous to and consistent with the variations illustrated in example 10b. In addition, other conjugates can be prepared using DOPA-containing polymers prepared by enzymatic conversion of tyrosine residues therein. Other techniques well-known in the field of peptide synthesis can be used with good effect to provide other desired protein sequences, peptide conjugates and resulting adhesive/anti-fouling effects.

### Example 12a

Gold surfaces were modified by adsorption of MPEG-DOPA or mPEG-MAPd (2 k, 5 k) from solution in DCM or phosphate-buffered saline (PBS; pH=3, 7.4, and 11) at polymer concentrations ranging from 0.1 -75 mg/ml. Substrates were placed in a vial and immersed in mPEG-DOPA or mPEG-MAPd solution for up to 24 hours without agitation. Upon removal from solution, substrates were rinsed with the appropriate solvent (DCM or DI H₂O) to remove unbound polymer, and dried in vacuo. For comparison, identical surface modifications were performed using PEG-monomethylether (mPEG-OH, M_{w=}5000). Alternatively, a drop of solution containing mPEG-DOPA or mPEG-MAPd (10 mM in PBS, PEG molecular weight=2000) was incubated on a Au-coated glass coverslip (Au thickness ˜10 nm) for 30 min at 37° C., after which the surface of the coverslip was rinsed (3×) with PBS. Analysis of the modified surfaces by advancing/receding contact angle, XPS, and TOF-SIMS revealed the formation of a chemisorbed layer of mPEG-DOPA or mPEG-MAPd.

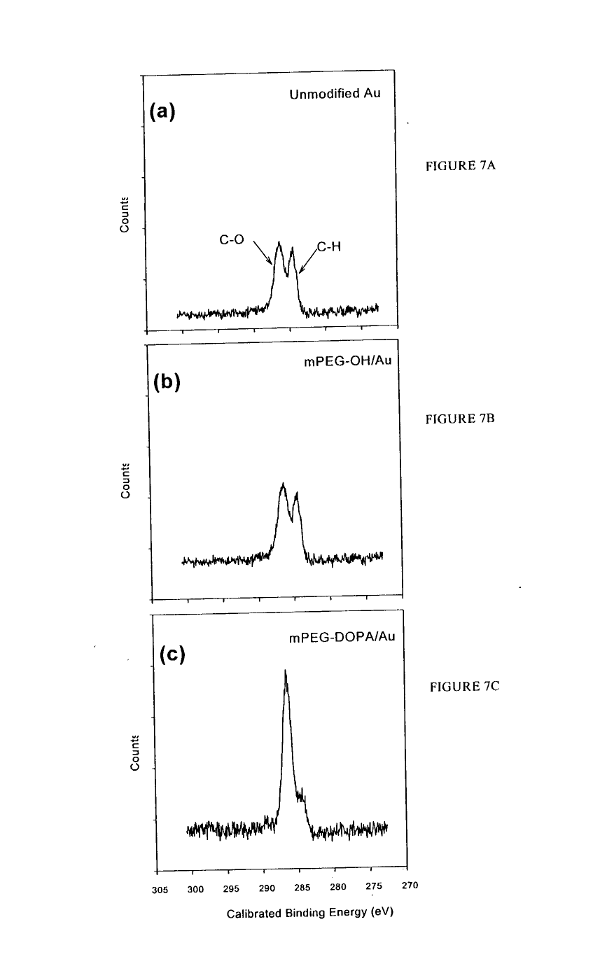

FIGS. 7A-C shows the XPS spectra for the unmodified, mPEG-OH modified, and mPEG-DOPA modified surfaces. As expected, the ether peak at 286.5 eV increased only slightly with the mPEG-OH treatment, while a dramatic increase was observed after adsorption of mPEG-DOPA, indicating a large presence of ether carbons. An ether peak from a pure PEG with the same binding energy has been reported in the literature. The smaller peak at 285.0 eV in FIG. 7 can be attributed to the aliphatic and aromatic carbons in the PEG and DOPA headgroup, as well as some hydrocarbon contamination resulting from the preparation/evacuation process.

Time-of-flight SIMS data corroborated the XPS findings. TOF-SIMS analysis was carried out on unmodified and mPEG-DOPA-modified Au substrates, as well as mPEG-DOPA powder and a gold substrate exposed to mPEG-OH. Data was collected from each substrate for ˜4 min.

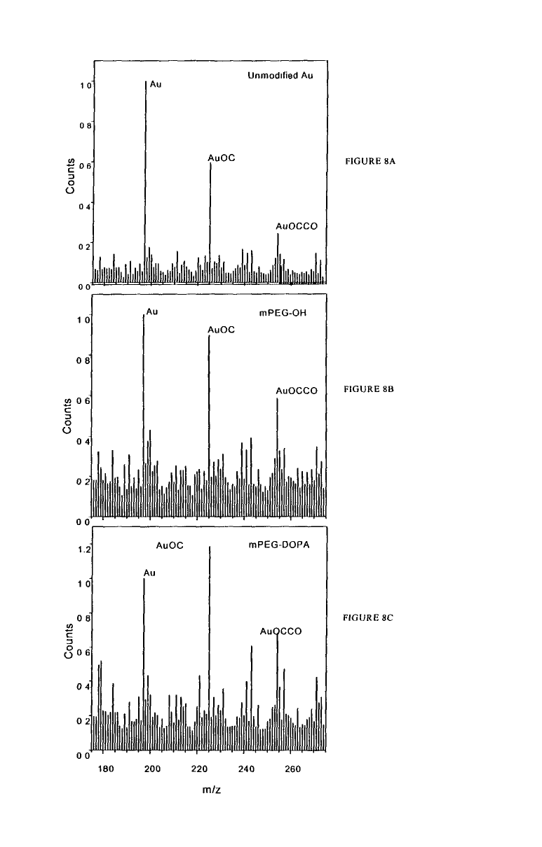

The positive ion spectrum of unmodified Au exhibits (CₙH₂ₙ₊₁)⁺ and (CₙH₂ₙ₋₁)⁺ peaks, typical for hydrocarbon contamination (data not shown). Additional minor contaminants were present, including NH₄⁺, Na⁺, and relatively small amounts of CₐH_{b}O_{c}⁺ species. Because of the process used to deposit the Au film, a peak for Cr was seen at m/z˜52, in addition to the Au peak at m/z˜196.9. Exposing the gold surfaces to mPEG-OH resulted in only modest increases in the peaks representing CₐH_{b}O_{c}⁺ PEG fragments, which are likely attributable to contamination or non-specific absorption of mPEG-OH. This is evidenced by the peaks at m/z˜225 (AuOC⁺) and 254 (AuOCCO⁺) which did not show dramatic increases when compared to substrates modified with mPEG-DOPA. (FIGS. 8A-C).

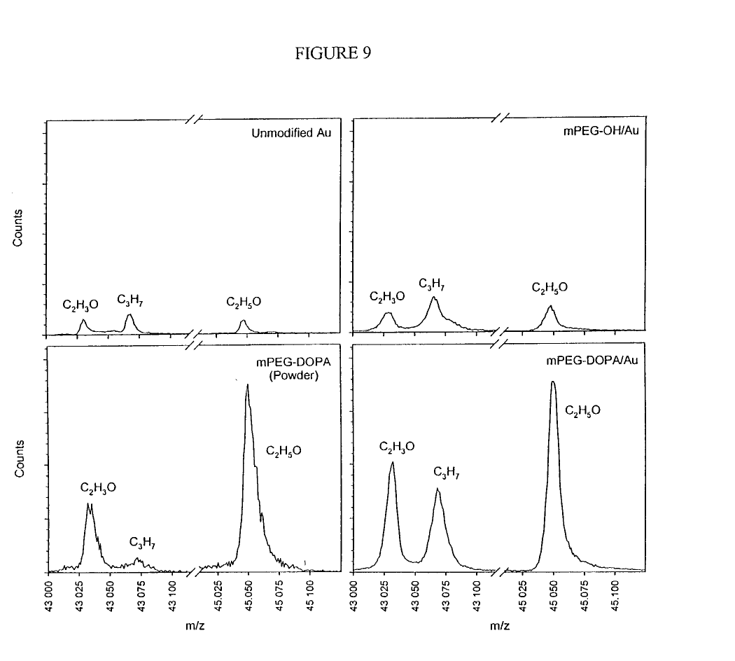

The positive ion spectrum of the Au surface modified with mPEG-DOPA was dominated by the presence of CₐH_{b}O_{c}⁺ peaks representing the adsorbed molecule. As illustrated in FIG. 9, the relative abundance of C₂H₃O⁺ and C₂H₅O⁺increased with respect to unmodified and mPEG-OH modified surfaces. There was also a dramatic increase in the relative abundance of C₃H₇⁺ (m/z˜43) and C₄H₅⁺ (M/z˜53), as well, which can likely be attributed to hydrocarbon contamination or the fragmentation of the t-butyl in the Boc protection group.

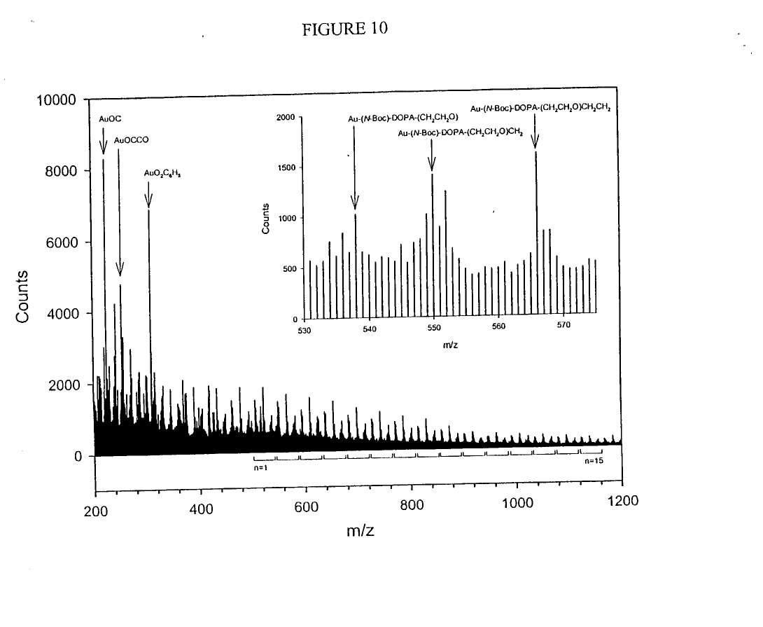

Perhaps the most notable feature of the positive ion spectrum of the PEGylated Au substrate were the patterned triplet repeats in the high mass range (FIG. 10). Each of these triplet clusters corresponds to an Au-DOPA-(CH₂CH₂O)ₙ fragment. When further resolved, each subcluster within the triplet represents the addition of CH₂, CH₂CH₂, or CH₂CH₂O, as each of these peaks is ˜14-16 amu apart. This repeat pattern was identifiable from n=0-15, beyond which the signal was below detectable limits.

In the negative ion spectra for the pristine Au surface, little of note was observed aside from the strong definable peaks for O⁻, HO⁻, and Auₙ⁻for n=1-3 (data not shown). There was a small amount of hydrocarbon contamination present at m/z˜13 (CH⁻), 24 (C₂H₂⁻), and 37 (C₃H⁻). The negative ion spectrum of the PEGylated Au surface was dominated by the peak for C₇H₁₁O₂⁺at m/z˜126.893. The presence of this peak at modest intensity in the spectrum of the mPEG-OH modified Au suggests that it represents a larger ethylene glycol fragment. The most interesting peaks lie in the high mass range (>200 m/z) and represent the coupling of catecholic oxygen to Au. The spectrum suggests that one Au atom can bind up to six oxygen atoms, corresponding to three DOPAs.

The contact angle data demonstrated a firm dependence on the character of the adsorption solvent used when modifying the gold films with mPEG-DOPA (data not shown). The surface modified in DCM showed a significantly lower θₐ than the unmodified surface (p<0.001) and the surfaces modified in all aqueous solutions (p<0.05). Generally speaking, as the pH of the aqueous solutions was increased, the hydrophilicity of the treated surfaces was decreased, indicating a diminished ability to PEGylate the surfaces, perhaps due to the propensity of DOPA to be oxidized to its less adhesive quinone form at elevated pH, an interpretation that is supported by previous studies that showed the unoxidized catechol form of DOPA is primarily responsible for adhesion.

### Example 12b

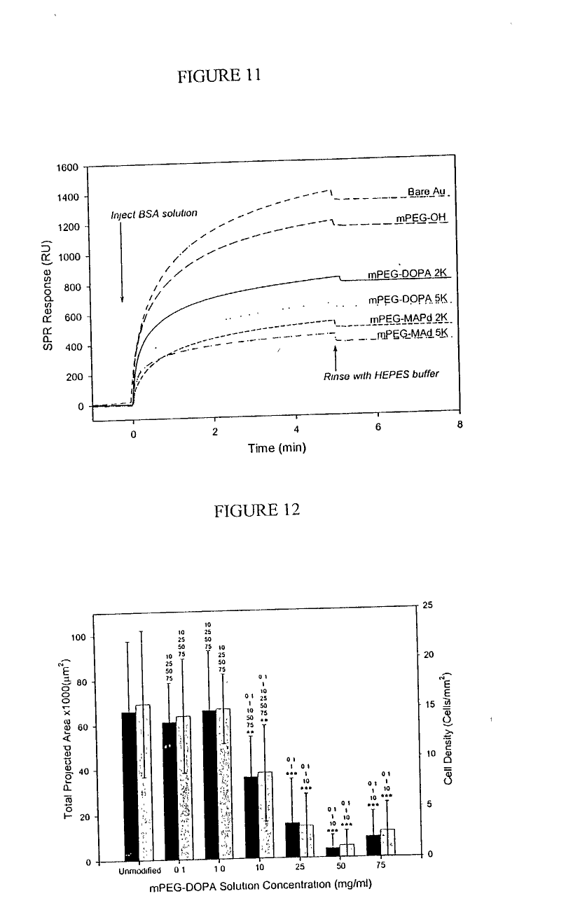

Protein adsorption and attachment/spreading of cells onto untreated and treated coverslips were evaluated as follows. Surface plasmon resonance (SPR) experiments demonstrated that the DOPA-containing polymers were rapidly bound to the gold surface and the resulting modified surfaces possessed an enhanced resistance to protein adsorption (FIG. 11). Protein adsorption onto mPEG-MAPd (5 k) modified gold was roughly 70% less than to the unmodified gold surface. Analysis of fibroblasts cultured on modified substrates showed a strong dependence of cell attachment on MPEG-DOPA concentration (FIG. 12), adsorption solvent, and modification time used during preparation of the PEG-modified substrates. Surfaces modified for 24 hours with >25 mg/ml MPEG-DOPA or mPEG-MAPd exhibited a statistically significant reduction in cell attachment and spreading (FIGS. 12-14). The mPEG-MAPd (5 k) modified gold surface exhibited a 97% reduction in total projected cellular area and a 91% reduction in the density of cells attached to the surface.

### Example 12c

The modification illustrated in example 12a, optionally varied as referenced in examples 10b and 11b, can be extended to other noble metals, including without limitation, silver and platinum surfaces. Such application can also be extended, as described herein, to include surface modification of any bulk metal or metal alloy having a passivating or oxide surface. For example, bulk metal oxide and related ceramic surfaces can be modified, as described herein. Such techniques can also be extended to semiconductor surfaces, such as those used in the fabrication of integrated circuits and MEMS devices, as also illustrated below in the context of nanoparticulate stabilization.

### Example 13

Silicate glass surfaces (glass coverslips) were modified by adsorption of mPEG-MAPd (2 k) from a 10 mM solution in water, using the method described in Example 12a. The cell density of NIH 3T3 cells attached to modified and unmodified glass surfaces were evaluated as described, above. Glass surfaces modified for 24 hours with mPEG-MAPd exhibited a 43% reduction in cell density compared to unmodified glass surfaces (Cell Density (cells/mm²): 75.5 +/−6.5 on unmodified glass; 42.7 +/−9.8 on mPEG-MAPd modified glass).

### Example 14a

To illustrate stabilization of metal oxides and, in particular, metal oxide nanoparticles, 50 mg of mPEG-DOPA (5 k) was dissolved in water (18MΩ-cm, Millipore) and combined with 1 mg of magnetite (Fe₃O₄) powder. Similar preparations were also prepared using a mPEG-NH₂ (5 k) (Fluka) and a mPEG-OH (2 k) (Sigma) as controls. Each of these aqueous solutions was sonicated using a Branson Ultrasonics 450 Probe Sonicator for one hour while being immersed in a 25° C. bath. The probe had a frequency of 20 kHz, length of 160 mm, and tip diameter of 4.5 mm. The sample was then removed and allowed to stand at room temperature overnight to allow any unmodified magnetite to precipitate out of solution. Suspensions prepared using the control polymers (mPEG-NH2 and mPEG-OH) rapidly precipitated to yield a brown solid and clear, colorless supernatant. In samples prepared using PEG-DOPA stabilized nanoparticles, the sample was clear and brown. The clear brown supernatant was isolated and dialyzed for three days in water using Spectra/Por® membrane tubing (MWCO:15,000). Following dialysis, the sample was lyophilized and stored under vacuum at room temperature until used.

### Example 14b

mPEG-DOPA stabilized nanoparticles were characterized by transmission electron microscopy (TEM), thermogravimetric analysis (TGA), fourier transform infrared spectroscopy (FTIR), and UV/vis spectroscopy. TEM results demonstrated that the majority of nanoparticles were of diameter of 5-20 nm (data not shown). TGA analysis of 0.4 mg of mPEG-DOPA stabilized magnetite indicated that the particles contain 17% by weight mPEG-DOPA (data not shown). Fourier transform infrared spectroscopy (FTIR) performed on untreated magnetite showed relatively little absorbance within the wavelength range from 4000-400(cm⁻¹), whereas the mPEG-DOPA treated nanoparticles exhibited absorption bands at 800-1600 cm⁻¹ and 2600-3200 cm⁻¹, confirming the presence of mPEG-DOPA.

### Example 14c

The dry PEG-DOPA stabilized magnetite nanoparticles readily dispersed in aqueous and polar organic solvents (e.g., dichloromethane) to yield clear brown suspensions that were stable for months without the formation of noticeable precipitates. Suspensions of mPEG-DOPA stabilized nanoparticles in various solvents were prepared by dispersing 1 mg of mPEG-DOPA treated magnetite in 1 ml of water (18MΩ-cm filtered using a Millex® AP 0.22 μm filter (Millipore)), DCM or Toluene. Suspensions were placed in a bath sonicator for ten minutes to disperse the nanoparticles. All three solutions were stable at room temperature for at least six months, whereas control suspensions of unmodified magnetite and magnetite stabilized by mPEG-OH or mPEG-NH2 precipitated out in less than 24 hours in each solvent.

### Example 14d

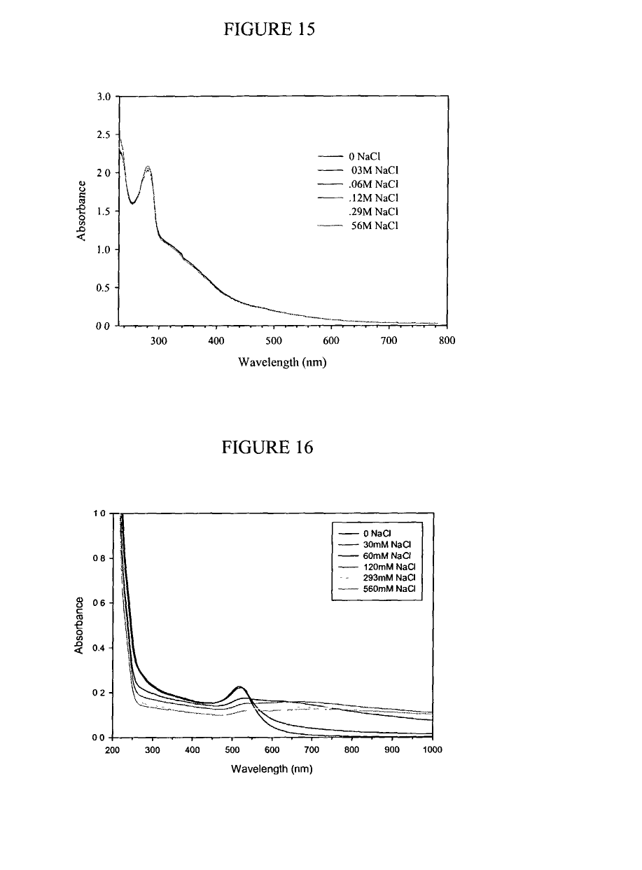

Suspensions of MPEG-DOPA stabilized nanoparticles were also found to be stable under physiologic concentrations of salt. To determine whether mPEG-DOPA could inhibit salt-induced nanoparticle aggregation, 0.3 mg of mPEG-DOPA treated magnetite was placed in a quartz cuvette and combined with 0.7 ml of water (18MΩ-cm filtered using a 0.25 μfilter). Aliquots of saturated NaCl solution (5μl, 10μl, 20 μl, 50 μl 100 μl) were sequentially added to the cuvette and allowed to stand for ten minutes before UV-VIS spectra were taken (FIG. 15). The absorbance spectra of mPEG-DOPA stabilized nanoparticles suspended in solutions containing increasing NaCl concentration were nearly identical, demonstrating that mPEG-DOPA is effective at stabilizing the nanoparticles and preventing aggregation. The peak centered at 280 nm is indicative of the catechol side chain of DOPA.

### Example 14e

The procedures and techniques illustrated in examples 14a-14d can be extended to various other metal oxide or ceramic nanoparticles, as would be understood by those skilled in the art made aware of this invention. Likewise, such applications of the present invention can further include use of a wide range of polymer-DOPA conjugates analogous to and consistent with those compositions and variations thereof described in examples 10b and 11b. As illustrated below in the preparation of semiconductor compositions, metal oxide or ceramic nanoparticles can be stabilized in situ upon formation in the presence of a polymer-DOPA conjugate of this invention.

### Example 15a

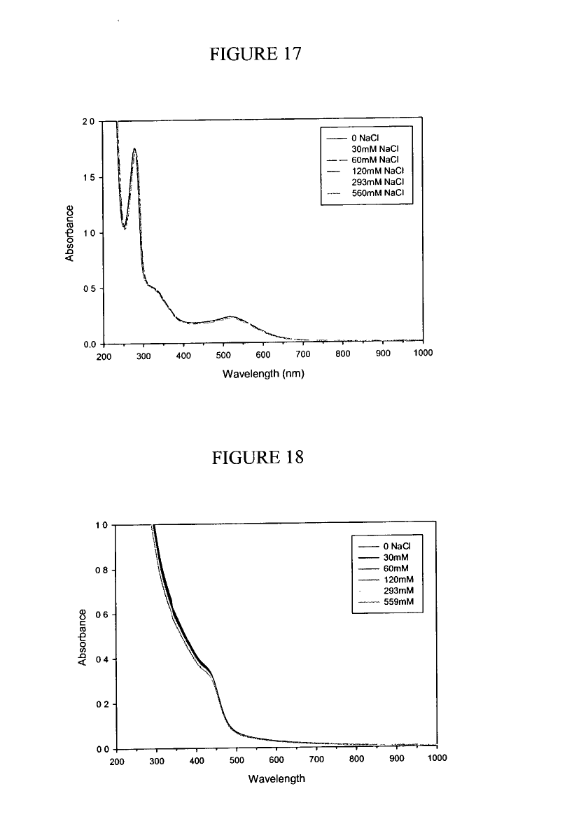

Demonstrating stabilization of metal nanoparticles, commercial gold colloid suspension (Sigma, particle size 5 or 10 nm) was placed inside dialysis tubing (MW cutoff of 8000 for 5 nm and 15000 for 10 nm) and dialyzed in ultrapure water for 2-3 days to remove the sodium azide present in the commercial preparation. The dialyzed suspensions were then placed into small glass vials and mPEG-DOPA added (10 mg/ml). The samples were allowed to stand at room temperature for approximately 2 days, after which the samples were again dialyzed to remove excess mPEG-DOPA. Untreated 10 nm Au nanoparticles were unstable in the presence of NaCl and aggregated (FIG. 16), whereas the treated Au nanoparticles remained stably suspended in the presence of aqueous NaCl (FIG. 17).

### Example 15b

Various other metal nanoparticles, including but not limited to, silver, platinum and the like can be stabilized as described in the preceding example. While stabilization was demonstrated using a representative conjugate composition of this invention, various other compositions can be prepared analogous to and consistent with the alternate embodiments described in examples 10b and 11b. Comparable results can be obtained by in situ formation of the stabilized nanoparticles synthesized from the corresponding metal precursor in the presence of a suitable, adhesive conjugate polymer of this invention.

### Example 16a

The data of this example demonstrates stabilization of semiconductor nanoparticles. CdS nanoparticles (quantum dots) were prepared by a standard method based on the slow mixing of dilute Cd(NO₃)₂ and Na₂S solutions. Fresh stock solutions (2 mM) of Cd(NO₃)₂ and Na₂S were prepared in nanopure water. The Na₂S solution was injected slowly into 50 ml of Cd(NO₃)₂ solution using a gastight syringe at a rate of 20 μl s⁻¹. The solution turned yellow with the addition of Na₂S, and after 2 mL of Na₂S was injected, a yellow precipitate appeared due to the aggregation of CdS nanoparticles. The CdS precipitate was isolated and dried for further use. Using the method described above for magnetite, the dry CdS powder was dispersed in a mPEG-DOPA solution by sonication to yield a clear yellow solution. The yellow aqueous suspension was stored in the dark for several months at room temperature without visible formation of precipitate. Control experiments performed in the absence of polymer and in the presence of mPEG-OH or mPEG-NH2 yielded yellow precipitate and a clear, colorless supernatant. MPEG-DOPA stabilized CdS nanoparticles remained stably suspended in the presence of aqueous NaCl (FIG. 18).

### Example 16b

The results of this example illustrate the in situ formation of stabilized semiconductor nanoparticles. CdS nanoparticles (quantum dots) were formed in the presence of mPEG-DOPA by slowly mixing dilute methanolic solutions of Cd(NO₃)₂ and Na₂S. Freshly prepared stock solutions (2 mM) of Cd(NO₃)₂ and Na₂S were prepared in methanol. 25 mg of mPEG-DOPA (PEG molecular weight=2000) was dissolved in 5 ml of 2 mM Cd(NO₃)₂ in methanol, then 5 ml of a 2 mM solution of Na₂S was added slowly with a syringe at a rate of 20 μl s⁻¹. The solution gradually turned yellow during the addition. No yellow precipitates were observed, and dynamic light scattering revealed particles with an average diameter of 2.5 nm. Control experiments performed in the absence of polymer or in the presence of mPEG-OH yielded yellow precipitate and a clear, colorless supernatant. Various other inorganic particulate substrates can be prepared, as would be understood by those skilled in the art, depending upon material choice and corresponding ionic substitution or exchange reaction, as carried out in the presence of an adhesive composition of the sort described herein.

### Example 16c

The polymeric conjugate compositions of this invention can also be used to stabilize a variety of other semiconductor materials. For instance, core-shell nanoparticles can be surface stabilized in accordance herewith.

### Example 17

The optimization experiments of this and subsequent examples were performed with mPEG-DOPA-5K. Several parameters were examined to optimize the adsorption of mPEG-DOPA onto gold from solution, including type and pH of solvent, time of adsorption, and mPEG-DOPA solution concentration. Cell attachment and spreading did not vary widely with adsorption solvent used. The number of cells on the substrates and their total projected area was not significantly different between DCM and three different aqueous solutions. The substrates adsorbed in neutral, basic, and organic mPEG-DOPA solutions all possessed significantly enhanced anti-fouling properties when compared to the unmodified substrate (p<0.01). Although no differences were observed in cell attachment and spreading between the solutions, the contact angle data would support the use of an organic solvent in an optimal modification protocol as a means to reduce catechol oxidation. Additionally, only the surface modified in DCM demonstrated significantly fewer cells on the surface and lower total projected cellular area.

### Example 18

Cell attachment and spreading showed a strong dependence on solution concentration of mPEG-DOPA (FIG. 12). Above 25 mg/ml MPEG-DOPA, significantly fewer cells attached and spread on the modified substrate than on the pristine gold surface (p<0.001) and the surface modified in a 10 mg/ml solution (p<0.05). Below 10 mg/ml, there were no differences in cell attachment and spreading compared to the unmodified substrate. There were no differences in cell attachment and spreading observed between surfaces modified in mPEG-DOPA solutions ranging from 25-75 mg/ml when compared to each other.

### Example 19

Fewer fibroblasts were observed to attach and spread with increasing duration of mPEG-DOPA adsorption, as well. Although cell attachment and spreading appeared to decrease with as little as 5 min of substrate modification, an adsorption time of 24h resulted in significantly fewer cells attaching and spreading on the PEGylated substrate than on the unmodified substrate (p<0.001) and substrates treated for shorter periods (p<0.05).

### Example 20

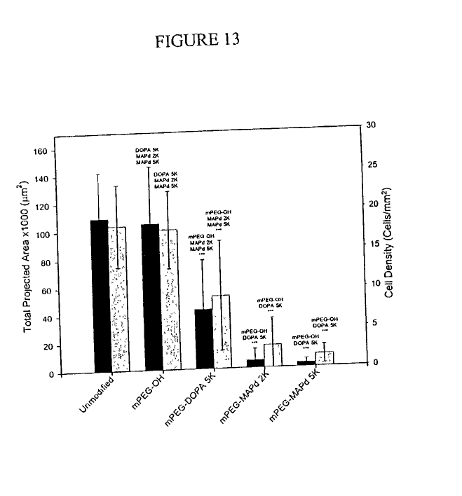

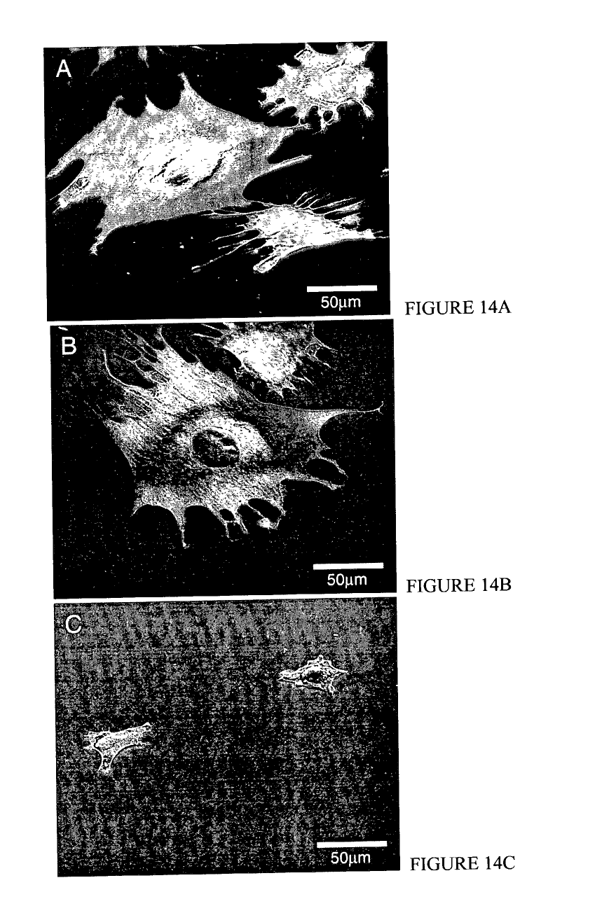

The morphology of fibroblasts cultured on both unmodified and PEG-modified surfaces was examined via electron microscopy (Hitachi 3500 SEM). Fibroblasts on unmodified Au and mPEG-OH-modified Au were generally flat and well spread, while those cultured on mPEG-DOPA modified Au were far less spread (FIGS. 14A-C). It should also be noted that on the mPEG-DOPA surface, a lower number of cellular processes were observed than in the others, structures which contribute to cell adhesion via integrins and focal adhesions. FIG. 13 illustrates the differences in attachment and spreading of fibroblasts on bare Au, mPEG-OH-treated Au, and Au modified with mPEG-DOPA 5K, mPEG-MAPd 2K, or mPEG-MAPd 5K under optimal conditions (50 mg/ml for 24h). The surfaces modified with DOPA-containing conjugates have significantly less cellular adhesion and spreading than either of the other two surfaces. The mPEG-MAP 5K modification, though, accounted for a 97% reduction in total projected cellular area and a 91% reduction in density of cells on the surface, a far greater reduction than that achieved by mPEG-DOPA 2K.

The differences in cellular adhesion and spreading between surfaces modified with DOPA- and MAPd-conjugated PEG in FIG. 13 can likely be attributed to the physical characteristics of the associated PEG adlayer. Analysis of the SPR results indicates that MAPd-PEGs form thicker, more robust adlayers with a higher concentration of PEG per unit area than do the DOPA-anchored PEGs of equivalent molecular weight. The thicker adlayers resulting from MAPd-mediated PEGylation are more successful in inhibiting protein adsorption and, in turn, cell adhesion.

While the principles of this invention have been described in connection with specific embodiments, it should be understood clearly that these descriptions are added only by way of examples and are not intended to limit, in any way, the scope of this invention. For instance, the present invention can enhance the adhesive properties of a wide variety of polymeric compositions, whether or not capable of hydrogelation. Likewise, the present invention can be used with various other synthetic techniques well known to those skilled in the art to functionally modify a particular polymeric component for a subsequent coupling and preparation of the corresponding DOPA conjugate. Other advantages, features and benefits will become apparent from the claims filed hereinafter, with the scope thereof as determined by their reasonable equivalents and as would be understood by those skilled in the art.

1. A biomimetic adhesive composition comprising a polymeric component and at least one catecholic component conjugated thereto, said polymeric component providing a surface active effect and comprising a poly(alkylene oxide).

2. The composition of claim 1 wherein said catecholic component comprises a moiety selected from the group consisting of DOPA, a DOPA-derivative and combinations thereof.

3. The composition of claim 2 wherein said moiety is a DOPA residue included within an amino acid sequence.

4. The composition of claim 3 wherein said sequence is the consensus decapeptide repeat sequence for the mussel adhesive protein of the blue mussel Mytilus edulis.

5. The composition of claim 1 wherein said polymeric component is a poly(alkylene oxide) co-polymer.

6. The composition of claim 5 wherein said polymeric component is a co-polymer of ethylene oxide and a hydrophobic co-monomer.

7. The composition of claim 6 wherein said hydrophobic co-monomer is selected from the group consisting of propylene oxide, lactic acid, glycolic acid and caprolactone.

8. The composition of claim 7 wherein said co-monomer comprises a hydrophobic block, and said polymeric component is a block co-polymer.

9. The composition of claim 8 wherein two catecholic components are conjugated to said polymeric component.

10. The composition of claim 9 admixed with a solvent.

11. The composition of claim 1 wherein said polymeric component comprises monomers selected from the group consisting of ethylene glycol, hyaluronic acid, a dextran and combinations thereof.

12. The composition of claim 11 wherein said polymeric component is a poly(ethylene glycol) conjugated to one catecholic component.

13. The composition of claim 12 wherein said catecholic component is selected from the group consisting of DOPA and an amino acid sequence including a DOPA residue.

14. The composition of claim 13 on a substrate.

15. A composite comprising a substrate and a biomimetic adhesive composition thereon, said composition comprising a polymeric component and at least one catecholic component conjugated thereto, said polymeric component comprising a poly(alkylene oxide).

16. The composite of claim 15 wherein said polymeric component comprises monomers selected from the group consisting of ethylene glycol, hyaluronic acid, a dextran and combinations thereof.

17. The composite of claim 16 wherein said polymeric component is a poly(ethylene glycol), and said polymeric component is conjugated to a catecholic component selected from the group consisting of DOPA, a DOPA-derivative and combinations thereof.

18. The composite of claim 17 wherein said catecholic component is a DOPA residue included within an oligopeptide.

19. The composite of claim 16 wherein said substrate comprises a material selected from the group consisting of noble metals, bulk metals, metal alloys and metallic compositions.

20. The composite of claim 19 wherein said substrate is a particulate.

21. The composition of claim 20 wherein said particulate is suspended in a liquid medium.

22. A method for in situ preparation of stabilized particulates, said method comprising:
(a) providing an admixture of a biomimetic adhesive composition and a first compound, said first compound a synthetic precursor to a predetermined particulate composition, said adhesive composition comprising a polymeric component and at least one catecholic component conjugated thereto, said polymeric component comprising a poly(alkylene oxide); and
(b) introducing a second compound to said admixture, said second compound another synthetic precursor to said particulate composition.

23. The method of claim 22 wherein said predetermined particulate composition is a semiconductor material.

24. The method of claim 23 wherein said semiconductor material is cadmium sulfide.

25. The method of claim 24 wherein said polymeric component comprises monomers selected from the group consisting of ethylene glycol, hyaluronic acid, a dextran and combinations thereof.

26. The method of claim 25 wherein said polymeric component is a poly(ethylene glycol) conjugated to a catecholic component selected from the group consisting of DOPA, a DOPA-derivative and combinations thereof.

27. A gelation system comprising a biomimetic adhesive composition in a liquid medium, said composition comprising a polymeric component and at least one catecholic component conjugated thereto, said polymeric component comprising a poly(alkylene oxide).

28. The system of claim 27 wherein said composition is substantially in solution at a first temperature and gels at a second temperature.

29. The system of claim 27 wherein said polymeric component is a poly(alkylene oxide) block co-polymer.

30. The system of claim 29 wherein said polymeric component is a co-polymer of ethylene oxide and a hydrophobic co-monomer.

31. The system of claim 30 wherein said hydrophobic co-monomer is selected from the group consisting of propylene oxide, lactic acid, glycolic acid and caprolactone.

32. The system of claim 31 wherein two catecholic components are conjugated to said polymeric component.

33. The system of claim 32 wherein each said catecholic component comprises a moiety selected from the group consisting of DOPA, a DOPA-derivative and combinations thereof.

34. A method for non-oxidative gelation of a DOPA-conjugated polymeric composition, said method comprising:
(a) providing an admixture of a polymeric composition and a liquid medium, said polymeric composition comprising a polymeric component and at least one DOPA component conjugated thereto, said polymeric component comprising a poly(alkylene oxide), and said DOPA component having substantial catecholic functionality; and
(b) increasing admixture temperature sufficient to gel said polymeric composition, said gelation substantially without oxidation of said catecholic functionality.

35. The method of claim 34 wherein said polymeric component is a block co-polymer having hydrophilic and hydrophobic blocks, and wherein increasing the length of said hydrophilic block relative to said hydrophobic block increases the gelation temperature of said polymeric composition.

36. The method of claim 34 wherein increasing the concentration of said polymeric composition in said liquid medium increases the gelation temperature of said polymeric composition.

37. The method of claim 34 wherein said polymeric component is a poly(alkylene oxide) block co-polymer.

38. The method of claim 37 wherein said polymeric component is a co-polymer of ethylene oxide and a hydrophobic co-monomer.

39. The method of claim 38 wherein two DOPA components are conjugated to said polymeric component, each said component selected from the group consisting of DOPA, a DOPA residue within an amino acid sequence, and a DOPA-derivative.
